SEQUENCE LISTING (43 sequences)

SEQ ID NO: 1 - protein, 6 aa, Artificial sequence
  Artificial peptide linker
GSGGGA

SEQ ID NO: 2 - protein, 12 aa, Artificial sequence
  Artificial peptide linker
GSGGGAGSGGGA

SEQ ID NO: 3 - protein, 18 aa, Artificial sequence
  Artificial peptide linker
GSGGGAGSGGGAGSGGGA

SEQ ID NO: 4 - protein, 24 aa, Artificial sequence
  Artificial peptide linker
GSGGGAGSGGGAGSGGGAGSGGGA

SEQ ID NO: 5 - protein, 10 aa, Artificial sequence
  Artificial peptide linker
GENLYFQSGG

SEQ ID NO: 6 - protein, 12 aa, Artificial sequence
  Artificial peptide linker
KPEPKPAPAPKP

SEQ ID NO: 7 - protein, 12 aa, Artificial sequence
  Artificial peptide linker
AEAAAKEAAAKA

SEQ ID NO: 8 - protein, 8 aa, Artificial sequence
  Artificial peptide linker
SACYCELS

SEQ ID NO: 9 - protein, 10 aa, Artificial sequence
  Artificial peptide linker
SGGGSSGGGS

SEQ ID NO: 10 - protein, 10 aa, Artificial sequence
  Artificial peptide linker
GGGGSGGGGS

SEQ ID NO: 11 - protein, 10 aa, Artificial sequence
  Artificial peptide linker
SSGGGSSGGG

SEQ ID NO: 12 - protein, 10 aa, Artificial sequence
  Artificial peptide linker
GGSGGGGSGG

SEQ ID NO: 13 - protein, 10 aa, Artificial sequence
  Artificial peptide linker
GSGSGSGSGS

SEQ ID NO: 14 - protein, 10 aa, Artificial sequence
  Artificial peptide linker
GGGSSGGGSG

SEQ ID NO: 15 - protein, 8 aa, Artificial sequence
  Artificial peptide linker
GLGGLAAA

SEQ ID NO: 16 - protein, 3 aa, Artificial sequence
  Artificial peptide linker
AAA

SEQ ID NO: 17 - protein, 5 aa, Artificial sequence
  Artificial peptide linker
GLGGL

SEQ ID NO: 18 - protein, 5 aa, Artificial sequence
  Artificial peptide linker
EAAAK

SEQ ID NO: 19 - protein, 3 aa, Artificial sequence
  Artificial peptide linker
GGG

SEQ ID NO: 20 - protein, 6 aa, Artificial sequence
  Artificial peptide linker
GSGSGS

SEQ ID NO: 21 - protein, 5 aa, Artificial sequence
  Artificial peptide linker
GGGSS

SEQ ID NO: 22 - protein, 15 aa, Artificial sequence
  Artificial peptide linker
GGGSSGGGSSGGGSS

SEQ ID NO: 23 - DNA, 5525 bp, Artificial sequence
  Plasmid vector
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattccgccgccaccatggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtctgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctggcggccgcgagggtgcagccgactgagagtatcgttcgtttccctaacattacgaacctatgtccgtttggagaggtatttaatgctaccagattcgcatcagtctatgcttggaacaggaaacggatatcaaactgcgtggccgactatagcgttctctacaacagtgcctcttttagcactttcaagtgctacggagtctccccaaccaagctaaacgacctgtgcttcacaaacgtctacgctgactcctttgtcatcagaggcgatgaggtgcggcaaatcgcacctggacaaactggaaaaatcgccgactataactataagctgcccgatgactttactggatgcgtgatcgcttggaattcaaacaatctcgacagtaaggtaggaggcaattacaactacctttatcggctcttccgaaaatcaaacctgaaaccctttgagcgtgatatctcgactgaaatttaccaagctggcagcactccttgcaacggagtggagggcttcaattgttactttcctctgcagagctacggattccaacctacaaacggggtaggctaccaaccctaccgagtggtggtgctgagctttgagctgctccatgctccagcaactgtatgcggccctaagaagtcaactaacctggttaagaataaatgcgtcaacttttagggatccagatctaacgacaaaacgacaaaacgacaaggcgccagatctggcgtttcgttttgtcgttttgtcgttagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 24 - protein, 483 aa, Artificial sequence
  Fusion protein
MAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLAAARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 25 - protein, 28 aa, SARS-CoV-2
KSVYYTSNPTTFHLDGEVITFDNLKTLL

SEQ ID NO: 26 - protein, 17 aa, SARS-CoV-2
KTYERHSLSHFVNLDNL

SEQ ID NO: 27 - protein, 21 aa, SARS-CoV-2
SSAKSASVYYSQLMCQPILLL

SEQ ID NO: 28 - protein, 28 aa, SARS-CoV-2
QAIASEFSSLPSYAAFATAQEAYEQAVA

SEQ ID NO: 29 - protein, 29 aa, SARS-CoV-2
FNSTYASQGLVASIKNFKSVLYYQNNVFM

SEQ ID NO: 30 - protein, 28 aa, SARS-CoV-2
YPTLNISDEFSSNVANYQKVGMQKYSTL

SEQ ID NO: 31 - protein, 15 aa, SARS-CoV-2
SVGPKQASLNGVTLI

SEQ ID NO: 32 - protein, 18 aa, SARS-CoV-2
MNVAKYTQLCQYLNTLTL

SEQ ID NO: 33 - protein, 16 aa, SARS-CoV-2
TATIPIQASLPFGWLI

SEQ ID NO: 34 - protein, 22 aa, SARS-CoV-2
FVTVYSHLLLVAAGLEAPFLYL

SEQ ID NO: 35 - protein, 11 aa, SARS-CoV-2
FKVSIWNLDYI

SEQ ID NO: 36 - protein, 11 aa, SARS-CoV-2
IVAAIVFITLC

SEQ ID NO: 37 - protein, 10 aa, SARS-CoV-2
VAAFHQECSL

SEQ ID NO: 38 - protein, 15 aa, SARS-CoV-2
FAYANRNRFLYIIKL

SEQ ID NO: 39 - protein, 23 aa, SARS-CoV-2
YKHWPQIAQFAPSASAFFGMSRI

SEQ ID NO: 40 - protein, 11 aa, SARS-CoV-2
SIVRFPNITNL

SEQ ID NO: 41 - protein, 17 aa, SARS-CoV-2
GVGYQPYRVVVLSFELL

SEQ ID NO: 42 - DNA, 6296 bp, Artificial sequence
  Plasmid vector
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattccgccgccaccatggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtctgagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaaggcctcggtggcctggcggccgcgaccgcaactattccaattcaggcctccctaccctttggctggctcatcggctcaggatctggatcagggagcgggtcttataagcattggccacaaatcgcccagttcgccccttccgcctcagcctttttcggcatgtcccggattggctcaggttctggttccgggtccgggagttttaactccacttacgcttcacagggcctagtcgcttccatcaagaactttaagtcagtgctctactatcagaacaacgtatttatgggcagcggcagcggctcaggatctggatcttatccaactttgaacatttcagacgagttctcctctaatgtagccaactatcagaaggtgggcatgcaaaagtacagcacccttggcagtggttcaggaagtggatctggcagctttgtgactgtttactctcacctcctcctagtcgctgccggcctcgaggccccattcctgtatctaggaagcggctccggtagtggaagcggatctgtcgcagcttttcaccaagagtgttccctaggcagtgggagtggttcaggcagtgggagcggcgtgggctatcagccttaccgggtcgtggtcttatccttcgagctacttggttccggctcagggagcggatctgggtcttccagcgctaaaagcgcttcagtatattactcccaactcatgtgccagcccatcctcctgctcggcagcggaagcggcagtggaagcggttctattgtggctgctatcgtttttatcaccctatgtggatctggcagcgggtctggaagtggctcctccatagtccgcttccctaatatcaccaacctcgggagcgggagcggttctggatctggatctcaggcaattgctagcgaattttcatccctaccctcttatgccgccttcgccacagcccaggaagcctatgagcaggcagtagctggatctggttccggcagtgggagtggttcatccgtgggccctaagcaagcctcactcaacggcgttaccctcatcggtagtggttctggaagcggcagtgggagcatgaacgtcgctaagtatacccagctctgccagtatctcaacaccctaaccctgggctccggatctgggtctggtagcggatcaaaaacatatgaaagacattcactctcccactttgttaatctcgacaaccttggctccggtagtggctctggctccggttcctttgcatatgctaaccgtaatcggtttctatatattatcaagctcggatcaggcagcggcagcggatctggaagttttaaggtctctatctggaatctcgattacatcggatctggcagcggaagtggctcagggtcgaaaagcgtctattacacttcaaaccccaccacctttcatctcgacggagaagtgatcacgtttgacaatctgaagacactcctttagggatccagatctaacgacaaaacgacaaaacgacaaggcgccagatctggcgtttcgttttgtcgttttgtcgttagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 43 - protein, 740 aa, Artificial sequence
  Fusion protein
MAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLAAATATIPIQASLPFGWLIGSGSGSGSGSYKHWPQIAQFAPSASAFFGMSRIGSGSGSGSGSFNSTYASQGLVASIKNFKSVLYYQNNVFMGSGSGSGSGSYPTLNISDEFSSNVANYQKVGMQKYSTLGSGSGSGSGSFVTVYSHLLLVAAGLEAPFLYLGSGSGSGSGSVAAFHQECSLGSGSGSGSGSGVGYQPYRVVVLSFELLGSGSGSGSGSSSAKSASVYYSQLMCQPILLLGSGSGSGSGSIVAAIVFITLCGSGSGSGSGSSIVRFPNITNLGSGSGSGSGSQAIASEFSSLPSYAAFATAQEAYEQAVAGSGSGSGSGSSVGPKQASLNGVTLIGSGSGSGSGSMNVAKYTQLCQYLNTLTLGSGSGSGSGSKTYERHSLSHFVNLDNLGSGSGSGSGSFAYANRNRFLYIIKLGSGSGSGSGSFKVSIWNLDYIGSGSGSGSGSKSVYYTSNPTTFHLDGEVITFDNLKTLL

(19) United States
(12) Patent Application Publication
(10) Pub. No.: US 2025/0304626 A1
(43) Pub. Date: Oct. 2, 2025

(54) Vaccination targeting intracellular pathogens

(71) Applicant: Evaxion Biotech A/S, Hørsholm (DK)

(72) Inventors: Anders Bundgård Sørensen, Hørsholm (DK); Jens Kringelum, Hørsholm (DK); Christian Garde, Hørsholm (DK); Thomas Trolle, Hørsholm (DK); Michael Schantz Klausen, Hørsholm (DK); Christian Thygesen, Hørsholm (DK); Gry Persson, Hørsholm (DK); Jesper Grønlund, Hørsholm (DK)

(21) Appl. No.: 18/563,716

(22) PCT Filed: Dec. 13, 2021
(86) PCT No.: PCT/EP22/64157

§ 371 Date: Nov. 22, 2023

(30) Foreign Application Priority Data
May 26, 2021 (EP) 21175997.2
Aug. 26, 2021 (EP) 21193308.0

(51) Int. Cl. C07K 14/005; A61K 39/00; A61K 39/215; A61P 37/04; C07K 14/195

(52) U.S. Cl. CPC C07K 14/005; A61K 39/215; A61P 37/04; C07K 14/195; A61K 2039/55511; C07K 2319/00; C12N 2770/20022; C12N 2770/20034

(57) ABSTRACT

Novel fusion polypeptides comprising a B-cell epitope-rich region, which comprises at least one fragment of at least one surface exposed protein from an intracellular pathogen, and a T-cell epitope-rich region, which comprises at least 2 densely arranged groups of T-cell epitope hotspots comprising at least one CTL inducing amino acid sequence, where the epitope hotspots are derived from at least two non-identical proteins of said intracellular pathogen. Also disclosed are nucleic acids and vectors encoding the fusion polypeptides and pharmaceutical means and methods based on the fusion polypeptides, nucleic acids and vectors.

### CROSS REFERENCE TO RELATED APPLICATIONS

This is a national stage filing in accordance with 35 U.S.C. § 371 of PCT/EP2022/064157, filed May 25, 2022, which claims the benefit of the priority of European Patent Application No. 21175997.2, filed May 26, 2021, and European Patent Application No. 21193308.0, filed Aug. 26, 2021, the contents of each are incorporated herein by reference.

### REFERENCE TO AN ELECTRONIC SEQUENCE LISTING

The contents of the electronic sequence listing (4564169Sequencelisting_ST25.txt; Size: 35,106 bytes and Date of Creation: Sep. 5, 2024) is herein incorporated in its entirety.

### FIELD OF THE INVENTION

The present invention relates to immunology, in particular vaccine technology. More specifically the present invention relates to means and methods intended to provide improved vaccines designed to prevent or treat infections with intracellular pathogens.

### BACKGROUND OF THE INVENTION

In general, when attempting to induce immunity via vaccination against intracellular pathogens such as virus and certain other infectious agents, it is as a rule desirable and/or necessary to ensure that specific cellular immunity against the pathogen is induced in the vaccinated individual, and the aim is to at least induce specific cellular immunity which enables CD8⁺ lymphocytes (also known as “cytotoxic T-lymphocytes”, “cytotoxic T-cells”, or abbreviated “CTLs”) to target infected cells and ultimately kill these. This is a consequence of the intracellular location of the pathogen while it replicates; while a humoral response (an antibody response) induced against an intracellular pathogen may to some extent prevent disease progression or in rare case initial infection, for instance by blocking the pathogen when it is present in the extracellular phase or by activating and stimulating NK cells when binding to cell surface exposed antigen, this is often not sufficient as a means for disease prophylaxis or therapy.

In addition, induction of both specific humoral and cellular immunity depends—at least in part on effective induction of CD4⁺ T helper cells that are activated by recognizing fragments of the pathogen's protein when presented by MHC Class-II molecules on the surface of professional antigen presenting cells (B-cells, dendritic cells, and macrophages).

These activated CD4+ cells in turn facilitate CD8+ T cell and B cell expansion through the release of stimulating cytokines and in the case of B cells through direct interaction with MHCII:peptide complex on the surface of these cells.

As a consequence, induction of effective specific immunity against an intracellular pathogen with a vaccine will optimally require that the vaccine agent can induce effective cellular immunity (both specifically reacting CD4⁺ and CD8⁺ cells) and humoral immunity. This is however far from always the case.

As an example, several vaccines have been and are currently being developed against SARS-CoV-2 (the pathogenic agent causing COVID-19), but many focus on the same single antigen from the virus, the so-called spike protein. The fact that such vaccines are based on one single surface exposed antigen may potentially lead to suboptimal CD4⁺/CD8⁺ activation by the vaccine, a problem which in turn can reduce the observed potency of the humoral response induced by the vaccine.

### OBJECT OF THE INVENTION

It is an object of embodiments of the invention to provide for vaccine agents and related therapeutic or prophylactic approaches, which adequately address infections of intracellular pathogens and multiple strains therein.

### SUMMARY OF THE INVENTION

The present inventors have come to the conclusion that in order to increase the efficacy of vaccines targeting intracellular pathogens, a number of conditions should be met: First of all, it is relevant—as with a number of existing vaccines—to be able to induce specific antibodies that can interfere with the ability of the infectious agent to infect cells and that it therefore is of high relevance to include B-cell epitopes from surface-exposed protein that e.g. serve as viral membrane fusion proteins (MFP) or as binding partners for receptors on the cells that become infected. It is in this regard relevant to consider the inclusion of multiple fragments or epitopes of such surface-exposed proteins from many strains and/or serotypes to increase the breadth of infectious agents covered by the vaccine.

Second, it is relevant that the vaccine agent comprises a sufficient number of T-cell epitopes (both MHC Class I and MHC Class II binding epitopes), so as to ensure adequate and sufficient CD4⁺ and CD8⁺ immunity. The aim of the generated CD4+ response is to provide Tₕₑₗₚₑᵣ cell support for an enhanced/improved generation of CD8+ effectors cells, and for providing further enhancement of the antibody response specific to the surface exposed proteins. The combination of these two elements will in consort ensure a more efficient neutralization of the infectious agent.

In addition, since the variability between strains and serotypes of the same infectious agent at the T-cell epitope level can be quite extensive without there being a large variability in the B-cell epitopes (which are by nature 3 dimensional structures that “fit” the binding sites of antibodies and B-cell receptors and which by nature are only relevant in so far they are exposed to the extracellular environment in the infected individual), it is relevant to include T-cell epitopes from a plurality of proteins from at least the most relevant serotypes and strains of the pathogen as this will ensure that the T-cell immunity induced is broad-spectred.

Finally, it is also relevant to construct the vaccine agents in a manner, which renders it uncomplicated to exchange epitopes over time without having to completely redesign the genetic tools used for the production of the vaccine agent.

The present inventors have identified strong MHCI/II ligands across the whole genome of selected intracellular pathogens and picked the best binders while ensuring to sample from as many proteins as possible. Nucleic acid sequences encoding epitopes from these two rounds of selection have been combined into “poly-epitope strings on a bead”-encoding design with encoded interspersed linkers, inserted into surface-exposed proteins of the target intercellular pathogen such as MFPs or suitable carrier proteins to enhance their expression in mammalian cells. The final constructs have then been inserted in an expression vector and subsequently tested in vaccination studies in mice.

So, in a 1^{st} aspect the present invention relates to a fusion polypeptide comprising i) a B-cell epitope-rich region, which comprises at least one fragment of at least one surface exposed protein from an intracellular pathogen, and ii) a T-cell epitope-rich region, which comprises at least 2 densely arranged groups of T-cell inducing amino acid sequences (epitope hotspots) comprising at least one CTL inducing amino acid sequences, where the epitope hotspots are derived from at least two non-identical proteins of said intracellular pathogen, wherein i) and ii) are directly fused to each other or indirectly fused to each other via linking amino acid sequences, and wherein B-cell epitopes and T-cell epitopes in said regions are derived from the intracellular pathogen.

In a 2^{nd} aspect, the present invention relates to a nucleic acid fragment which a) encodes the fusion polypeptide of the first aspect of the invention or any embodiments of the 1^{st} aspect, which are disclosed herein, or b) encodes at least or exactly two polypeptides, of which one comprises or consists essentially of a B-cell epitope-rich region (i) disclosed in the context of the 1^{st} aspect of the invention or any embodiments of the 1^{st} aspect of the invention, which are disclosed herein, and of which one other comprises a T-cell epitope-rich region (ii) disclosed in the context of the 1^{st} aspect of the invention and any embodiments of the 1^{st} aspect, which are disclosed herein.

In a 3^{rd} aspect, the present invention relates to an expression vector, which comprises the nucleic acid fragment of the 2^{nd} aspect of the invention or any embodiments of the 2^{nd} aspect, which are disclosed herein.

In a 4^{th} aspect, the present invention relates to a pharmaceutical composition comprising a fusion polypeptide of the 1^{st} aspect of the invention of any embodiments of the 1^{st} aspect, which are disclosed herein, and further comprising a pharmaceutically acceptable carrier, vehicle, diluent, and/or excipient, and optionally an immunological adjuvant.

In a 5^{th} aspect, the present invention relates to a pharmaceutical composition comprising at least two polypeptides that can be encoded by the nucleic acid fragment, option b, of the 2^{nd}aspect of the invention or any embodiments of the 2^{nd} aspect, option b, which are disclosed herein, and further comprising a pharmaceutically acceptable carrier, vehicle, diluent, and/or excipient, and optionally an immunological adjuvant.

In a 6^{th} aspect, the present invention relates to a pharmaceutical composition comprising a nucleic acid fragment of the 2^{nd} aspect of the present invention or any embodiments of the 2^{nd} aspect, which are disclosed herein, or an expression vector of the 3^{rd} aspect of the invention or any embodiments of the 3^{rd} aspect, which are disclosed herein, and further comprising a pharmaceutically acceptable carrier, vehicle, diluent, and/or excipient, and optionally an immunological adjuvant

In a 7^{th} aspect, the present invention relates to a pharmaceutical composition comprising
a) a first nucleic acid fragment encoding a polypeptide, which comprises or consists essentially of a B-cell epitope-rich region (i) as disclosed in the context of the 1^{st} aspect of the invention or any embodiments of the 1^{st} aspect, which are disclosed herein, and a second nucleic acid fragment encoding a polypeptide which comprises or consists essentially of a T-cell epitope-rich region (ii) as disclosed in the context of the 1^{st} aspect of the invention or any embodiments of the 1^{st} aspect, which are disclosed herein; or
b) at least 2 expression vectors, of which one comprises the first nucleic acid fragment defined in a) and further comprises the second nucleic acid fragment defined in a); and further comprising a pharmaceutically acceptable carrier, vehicle, diluent, and/or excipient, and optionally an immunological adjuvant.

In an 8^{th} aspect, the present invention relates to a method of inducing or enhancing an immune response against an intracellular pathogen in an animal, preferably a human being, the method comprising administering to an individual in need thereof an effective and pharmaceutically acceptable amount of a fusion polypeptide of the 1^{st} aspect of the present invention or any embodiments of the 1^{st} aspect, which are disclosed herein, the nucleic acid fragment of the 2^{nd} aspect of the invention or any embodiments of the 2^{nd} aspect, which are disclosed herein, an expression vector of the 3^{rd} aspect of the invention or any embodiments of the 3^{rd} aspect, which are disclosed herein, or a pharmaceutical composition of any one of the 4^{th}-7^{th} aspects of the present invention of any embodiments of any of the 4^{th}-7^{th} aspects of the invention, which are disclosed herein.

Finally, in aspects related to the 8^{th} aspects, the present invention relates to the fusion polypeptide of the 1^{st} aspect of the present invention or any embodiments of the 1^{st} aspect, which are disclosed herein, the nucleic acid fragment of the 2^{nd} aspect of the invention or any embodiments of the 2^{nd} aspect, which are disclosed herein, an expression vector of the 3^{rd} aspect of the invention or any embodiments of the 3^{rd} aspect, which are disclosed herein, or a pharmaceutical composition of any one of the 4^{th}-7^{th} aspects of the present invention of any embodiments of any of the 4^{th}-7^{th} aspects of the invention, which are disclosed herein, for use as a medicament, and in particular for use in a method of the 8^{th} aspect of the invention or any embodiments of the 8^{th} aspect, which are disclosed herein.

### LEGENDS TO THE FIGURE

FIG. 1: Schematic representation of the immune responses induced after administration of a nucleic acid construct of the present invention, in this case exemplified by a plasmid. The mechanisms behind the simultaneous induction of cellular and humoral immunity are depicted in simplified form. (1) Cell entry. (2) Antigen translation and export. (3) Targeting for internalization by the antigen presenting cell targeting unit (APCt). (4) Antigen processing and display of MHC I/II ligands. (5) Activation by Tₕₑₗₚₑᵣ cells by APCs. (6) B cell receptor recognition and internalization. (7) B cell processing and display of MHC II ligands. (8) Activation of B cells by Tₕₑₗₚₑᵣ cells. (9) Release of activating cytokines. (10) Further activation of B cells by T_{H} cytokines. (11) Activation of CD8⁺ T cells by APCs. (12) Further activation of CD8⁺ T cells by T_{H} cytokines.

FIG. 2: Conceptual high-level view of the vaccine design concept.

FIG. 3: Detailed overview of the design concepts and the possible elements that may be combined in any order or combination between elements i, ii, iii and iv. (i) The B cell rich region. Various preferred design concepts are illustrated in a) a single membrane fusion protein (MFP) or similar relevant surface exposed target, b) the joining of multiple MFPs (n) or similar from the same pathogen, multiple strains or pathogens. c) the in a or b mention design with supportive CD4⁺ ligands as Hotspots on a string with/without padding regions and linkers. d) the design in c where said CD4 ligands are integrated as part of the MFPs or similar targets. (ii) The T cell rich region with a number of these Hotspots that may a) be joined by simple linkers (intra), b) have padded regions, c) or have both in combination. (iii) The Antigen Presenting Cell targeting unit. (iv) The multimerization domain preferably consisting of a) a fragment from the human IgG3 Heavy chain or b) fragment of the T4 Fibritin foldon domain.

FIG. 4: Plasmid map of circular plasmid P0055.

FIG. 5: Plasmid map of circular plasmid P0057.

FIG. 6: Schematic presentation of T cell Hotspot sequences encoded in plasmid P0057.

FIG. 7: Schematic explanation to vaccine designs shown in FIGS. 8-16.

FIG. 8: Schematic presentation of Proof-of-concept designs according to the present invention.

FIG. 9: Schematic presentation of RBD-Fragment designs optimized by addition of His-tags to P0055 and position of the multimerization domain.

FIG. 10: Schematic presentation of optimization of RBD-Fragment designs by mutagenesis of potential problematic Cys538 to Ser.

FIG. 11: Schematic presentation of SARS-COV-2 spike S1 domain designs with/without a T-cell component, using T4 as the trimerization domain.

FIG. 12: Schematic presentation designs using SARS-COV-2 full-length spike protein with proline stabilization (2P), removal of the transmembrane region (ΔCT), and addition of T4-foldon domain.

FIG. 13: Schematic presentation of chimeric designs with the N-terminal and C-terminal domains from different coronavirus strains.

FIG. 14: Schematic presentation of designs with CD4⁺ T cell epitopes (black squares) grafted into the S2 domain of SARS-CoV-2 full-length spike by multiple sequence alignment (MSA) and/or BIFROST.

FIG. 15: Schematic presentation of different T cell Hotspot designs.

FIG. 16: Schematic presentation of designs with different linker types (grey squares) between encoded T cell Hotspots (black squares).

FIG. 17: Bar graphs showing anti-RBD IgG endpoint titer levels (A) and neutralizing effect of the antibodies (B) upon immunization of BALB/c mice using intramuscular injection. 1: P0055 (0 wk); 2: P0055 (0,4 wk); 3: P0055 (0, 2, 4 wk); 4: P0055 (0, 1, 2, 3, 4 wk); 5: P0055⁺ P0058 (0, 2, 4 wk); 6: rRBD+Al; 7: Al; 8: P0053 Mock (0, 1, 2, 3, 4).

FIG. 18: Bar graphs showing anti-RBD IgG endpoint titer levels (A) and neutralizing effect of the antibodies (B) upon immunization of C57BL/6 mice using intramuscular injection.

FIG. 19: Bar graphs showing anti-RBD IgG endpoint titer levels (A) and neutralizing effect of the antibodies (B) upon immunization of C57BL/6 mice using intramuscular injection (IM) lone or followed by electroporation (EP).

FIG. 20: (A) Heatmap showing IFN-γ responses in ELISPOT assays performed on cells from C57BL/6 or BALB/c mice stimulated with peptides. (B) Bar graph showing IFN-γ responses in ELISPOT assays on cells from K18-hACE2 C57BL/6J mice. (C) Survival curve of K18-hACE2 C57BL/6J mice upon infection with SARS-CoV-2.

FIG. 21: Bar graphs showing IFNγ release in ELISPOT performed on murine T-cells against (A) peptide pools covering the RBD and (B) individual peptides contained in RBD peptide pool 3.

### DETAILED DISCLOSURE OF THE INVENTION

### Definitions

The term “polypeptide” is in the present context intended to mean both short peptides of from 2 to 10 amino acid residues, oligopeptides of from 11 to 100 amino acid residues, and polypeptides of more than 100 amino acid residues. Further-more, the term is also intended to include proteins, i.e. functional biomolecules comprising at least one polypeptide; when comprising at least two polypeptides, these may form complexes, be covalently linked, or may be non-covalently linked. The polypeptide (s) in a protein can be glycosylated and/or lipidated and/or comprise prosthetic groups.

A “fusion polypeptide” has its usual meaning in molecular biology and denotes a polypeptide constitute by at least two (poly)peptides mutually linked via a peptide bond involving the C-terminus of one polypeptide and the N-terminus of another polypeptide, where the at least 2 polypeptides are not naturally linked to each other in the same sequence.

The term “subsequence” means any consecutive stretch of at least 3 amino acids or, when relevant, of at least 3 nucleotides, derived directly from a naturally occurring amino acid sequence or nucleic acid sequence, respectively.

The term “amino acid sequence” is the order in which amino acid residues, connected by peptide bonds, are arranged in the chain in peptides and proteins.

A “B-cell epitope” is a molecule or part of a molecule, which bind specifically to the antigen binding pocket in an antibody or in a B-cell receptor. In the context of polypeptides or proteins, these can include B-cell epitopes, which are either constituted by a stretch of amino acids, i.e. a linear epitope, or constituted by amino acids from different locations in one or more polypeptide chains, i.e. an assembled topographic epitope.

A “T-cell epitope” is a peptide (normally a fragment of a larger polypeptide) which 1) binds and can be presented to T-cells on a relevant cell surface by an MHC molecule, and 2) which can be recognized by a T-cell receptor when presented by the MHC molecule. An MHC Class I T-cell epitope is typically 8-10 amino acid residues in length and is presented by MHC Class I molecules on the surface of nucleated cells and in turn the complex of the peptide and the MHC Class I molecule is recognized by CD8⁺ cytotoxic T lymphocytes (also termed cytotoxic T-cells or CTLs). An MHC Class II T-cell epitope is typically 15-25 amino acid residues in length (but can be as short as 13 amino acid residues and no defined upper length exists) and is presented by MHC Class II molecules on the surface of professional antigen presenting cells and in turn the complex of the peptide and the MHC Class I molecule is recognized by CD4⁺ T lymphocytes (also termed T-helper lymphocytes, T-helper cells or Th cells.).

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Sequence identity” is in the context of the present invention determined by comparing 2 optimally aligned sequences of equal length (e.g. DNA, RNA or amino acid) according to the following formula: (N_{ref}−N_{dif})·100/N_{ref}, wherein N_{ref} is the number of residues in one of the 2 sequences and N_{df} is the number of residues which are non-identical in the two sequences when they are aligned over their entire lengths and in the same direction. So, two sequences 5′-ATTCGGAAC-3′ and 5′-ATACGGGAC-3′ will provide the sequence identity 77.8% (N_{ref}=9 and N_{dlf}=2). It will be understood that such a sequence identity determination requires that the two aligned sequences are aligned so that there are no overhangs between the two sequences: each amino acid in each sequence will have to be matched with a counterpart in the other sequence.

An “assembly of amino acids” means two or more amino acids bound together by physical or chemical means.

The “3D conformation” is the 3-dimensional structure of a biomolecule such as a protein. In monomeric polypeptides/proteins, the 3D conformation is also termed “the tertiary structure” and denotes the relative locations in 3-dimensional space of the amino acid residues forming the polypeptide.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “linker” is an amino acid sequence, which is introduced between two other amino acid sequences in order to separate them spatially. A linker may be “rigid”, meaning that it does substantially not allow the two amino acid sequences that it connects to move freely relative to each other. Likewise, a “flexible” linker allows the two sequences connected via the linker to move substantially freely relative to each other. In the fusion proteins, which are part of the present invention, both types of linkers can be useful.

Other linkers of interest are listed in the following table:

| | | Between or inside elements |
|---|---|---|
| Type | Sequence | I-V (if relevant) |
| Flexible | GSGGGA | Between |
| | (SEQ ID NO: 1) | |
| Flexible | GSGGGAGSGGGA | Between |
| | (SEQ ID NO: 2) | |
| Flexible | GSGGGAGSGGGAGSGGGA | Between |
| | (SEQ ID NO: 3) | |
| Flexible | GSGGGAGSGGGAGSGGGAGSGGGA | Between |
| | (SEQ ID NO: 4) | |
| Flexible | GENLYFQSGG | Between or inside |
| | (SEQ ID NO: 5) | |
| Rigid | KPEPKPAPAPKP | Between or inside |
| | (SEQ ID NO: 6) | |
| Rigid | AEAAAKEAAAKA | Between or inside |
| | (SEQ ID NO: 7) | |
| Rigid | SACYCELS | Between or inside |
| | (SEQ ID NO: 8) | |
| Flexible | SGGGSSGGGS | Inside T-cell rich epitopes |
| | (SEQ ID NO: 9) | |
| Flexible | GGGGSGGGGS | Inside T-cell rich epitopes |
| | (SEQ ID NO: 10) | |
| Flexible | SSGGGSSGGG | Inside T-cell rich epitopes |
| | (SEQ ID NO: 11) | |
| Flexible | GGSGGGGSGG | Inside T-cell rich epitopes |
| | (SEQ ID NO: 12) | |
| Flexible | GSGSGSGSGS | Inside T-cell rich epitopes (P0067) |
| | (SEQ ID NO: 13) | |
| Flexible | GGGSSGGGSG | Inside IgG3 HC elements (P0055) |
| | (SEQ ID NO: 14) | |
| Flexible | GLGGLAAA | Between IgG3 HC and B-cell rich |
| | (SEQ ID NO: 15) | antigen (p0055) |
| Flexible | AAA | Between IgG3 HC and B-cell rich |
| | (SEQ ID NO: 16) | antigen (p0055) |
| Flexible | GLGGL | Between IgG3 HC/T4 and T-cell rich |
| | (SEQ ID NO: 17) | epitopes (p0067) |
| Rigid | EAAAK | Between APC Targeting and B-cell rich |
| | (SEQ ID NO: 18) | antigen (p0070) |
| Flexible | GGG | Inside T-cell rich epitopes (p00XX) |
| | (SEQ ID NO: 19) | |
| Flexible | GSGSGS | Between B-cell rich antigen and |
| | (SEQ ID NO: 20) | T4/IgG3 HC (P0070) |
| Flexible | GGGSS | Between hinge region and CH3 domain |
| | (SEQ ID NO: 21) | |
| Flexible | GGGSSGGGSSGGGSS | Between hinge region and CH3 domain |
| | (SEQ ID NO: 22) | |

A “Pad region” is a number of amino acids added N and C terminally of an identified MHC-I or MHC-II ligand to facilitate proper processing and presentation of said ligands when the fusion proteins is taken up or produced in a target cell. Such pad regions can be derived from the proteome of the target intracellular pathogen, and in most cases directly adjacent to the identified ligand. Padded regions have been used in so-called “long synthetic peptide” strategies in neo epitope vaccines based on MHCI/II ligands (X) where additional non-ligand relevant amino acids (Y) have been included around the identified oncogenic mutation(M) e.g. YYYYYYYYXXXXXMXXXXXYYYYYYYY.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Hybridization under “stringent conditions” is herein defined as hybridization performed under conditions by which a probe will hybridize to its target sequence, to a detectably greater degree than to other sequences. Stringent conditions are target-sequence-dependent and will differ depending on the structure of the polynucleotide. By controlling the stringency of the hybridization and/or washing conditions, target sequences can be identified which are 100% complementary to a probe (homologous probing). Alternatively, stringency conditions can be adjusted to allow some mismatching in sequences so that lower degrees of similarity are detected (heterologous probing). Specificity is typically the function of post-hybridization washes, the critical factors being the ionic strength and temperature of the final wash solution. Generally, stringent wash temperature conditions are selected to be about 5° C. to about 2° C. lower than the melting point (Tm) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tm, which is also called the melting temperature. Formulas are available in the art for the determination of melting temperatures.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention substantially all will mount an immune response against the immunogen of the present invention.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterologous nucleic acid sequence

The term “expression vector” refers to a vector containing a nucleic acid sequence coding for at least part of a gene product capable of being transcribed. In some cases, when the transcription product is an mRNA molecule, this is in turn translated into a protein, polypeptide, or peptide.

### Specific Embodiments of the Invention

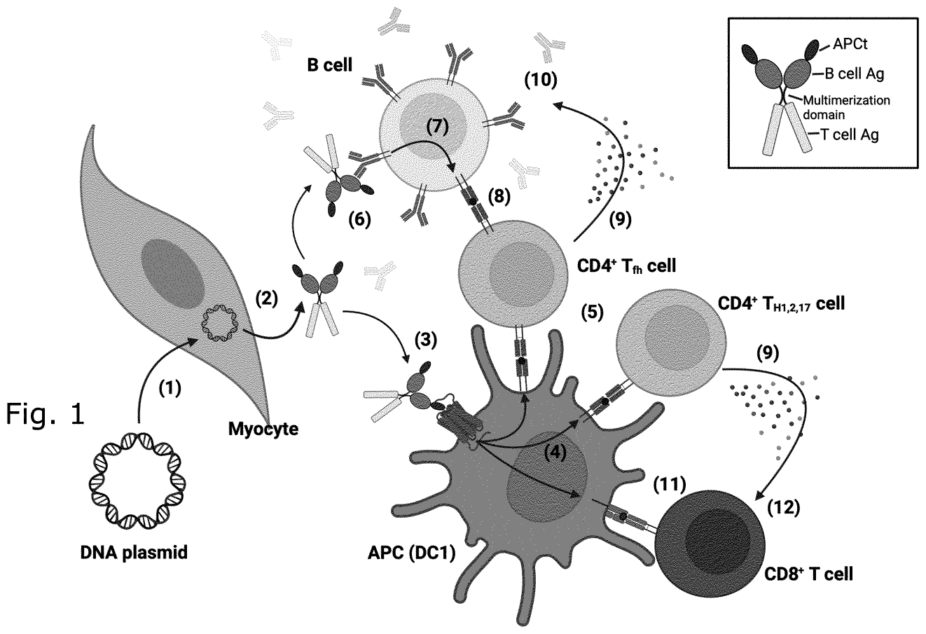

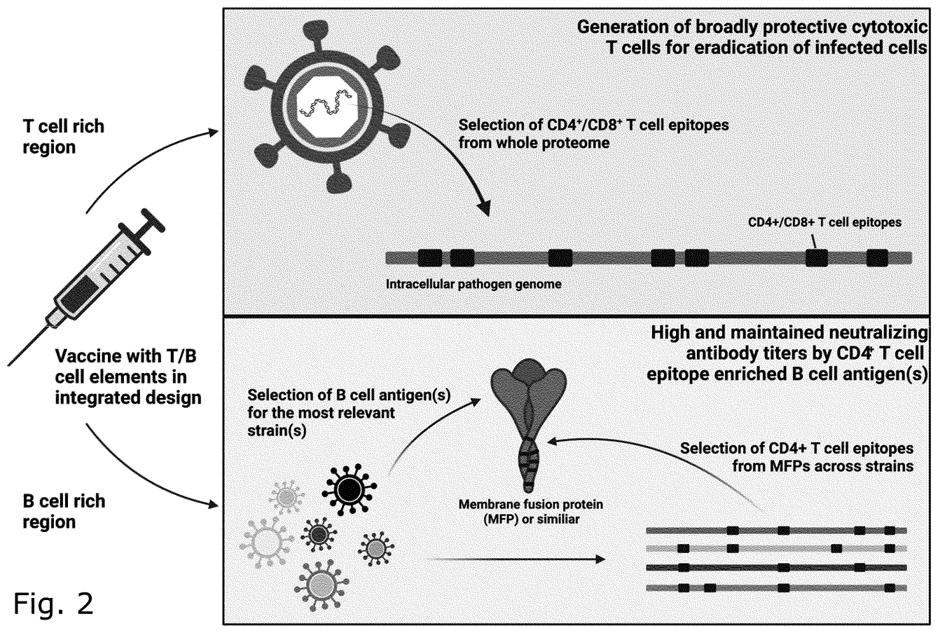

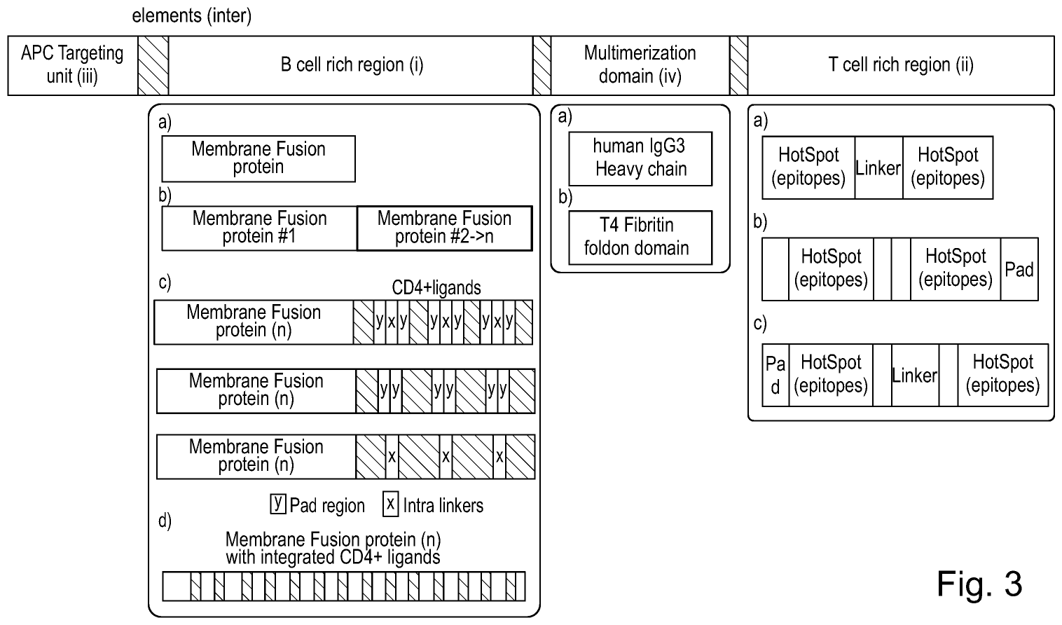

The immunologic events triggered by nucleic acid vaccines of the invention are set forth in FIG. 1, whereas the generic vaccine designs of the invention are set forth in FIG. 2 and in more detail in FIG. 3 is provided certain embodiments of the generic design. Reference is generally made to these 3 figures in the more detailed discussion below.

### 1^{st} Aspect of the Invention

In iFimportant embodiments, the fusion polypeptide of the 1^{st} aspect of the invention further comprises at least one amino acid sequence acting as a targeting unit for antigen presenting cells (an APC targeting unit) (the targeting unit is generally to by the roman numeral iii herein). Useful APC targeting units and nucleic acid constructs encoding immunogens that comprise such APC targeting units are disclosed in WO 2021/204911 and WO 2022/013277, both of which are incorporated by reference herein.

The APC targeting unit (iii) can consist of or comprise an antibody binding region with specificity for target surface molecules on antigen presenting cells, such as target surface molecules HLA, HLA-DP, CD14, CD40, or Toll-like receptor, such as Toll-like receptor 2.

Also, the APC targeting unit (iii) can consist of or comprise a ligand, such as soluble CD40 ligand; CLEC9A peptide ligand, DEC205, FLT3L, GM-CSF, natural ligands like chemokines, such as a chemokine of the CC chemokine family, such as any one selected from chemokine ligand 3, chemokine ligand 4, chemokine ligand 5, chemokine ligand 19, chemokine ligand 20, chemokine ligand 21, or similar; or a chemokine of the CXC chemokine family, such as any one selected from chemokine (C-X-C motif) ligand 1 (CXCL1), ligand 13 (CXCL13) or similar, such as RANTES or Chemokine ligand 3 (CCL3/MIP-1a) or CCL19; or bacterial antigens, such as flagellin or a part thereof.

Generally, the targeting units disclosed herein merely need to comprise the binding part of the ligand that interact with the target for the ligand. This also means that minor sequence variants (such as those known from allelic variants of the ligand) as well as fragments thereof can be used as a substitutes for the ligand itself. Hence, when discussing the use of a particular ligand, it will be understood that this also is intended to cover use of molecules that comprise or consist of: 1) the minimum binding fragment of the ligand, 2) minimum binding fragments of allelic variants of the ligand, and 3) minimum binding fragments of homologues of the ligand from other species, where the homologues share affinity for the targeted sequence with the ligand.

It is of particular relevance that the APC targeting unit (iii) is targeting mature dendritic cells (mDCs).

In certain embodiments, the APC targeting unit (iii) is selected from CCL19 and CCL21, such as the human forms. In related important embodiments, the APC targeting unit (iii) is one that targets the receptor CCR7. This has the consequence that minimal binding fragments of both CCL19 and CCL21 that bind CCR7 can be used instead of CCL19 and/or CCL21 in all embodiments disclosed herein—likewise sequence variants, such as allelic variants of CCL19 and CCL21 as well as CCR7-binding fragments thereof are also contemplated as alternatives that will serve the same purpose the CCL19 and CCL21 polypeptides disclosed herein.

In other embodiments, the APC targeting unit (iii) is selected from CCL3, CCL4, CCL5, CCL20, or XCL1, such as the human forms thereof. Also, functional fragments, allelic variants and their functional fragments, and the corresponding chemokines from various species and functional fragments thereof are within the meaning of the terms CCL3, CCL4, CCL5, CCL20, or XCL1.

In certain embodiments, the APC targeting unit (iii) is targeting immature dendritic cells (imDCs). In certain embodiments the APC targeting unit (iii) is targeting a receptor selected from CCR1, CCR3, CCR5, CCR6, and XCR1.

In certain embodiments, the APC targeting unit (iii) is CXCL13, such as the human forms. In related important embodiments, the APC targeting unit (iii) is one that targets the receptor CXCR5 on B cells belonging to both the B-1 and B-2 subsets and on the T follicular helper cells (Tfh) are a specialized subset of CD4+ T cells.

The APC targeting unit (iii) may as indicated above consist of or comprise an antibody binding region (i.e. the binding region of an antibody) with specificity for target surface molecules on antigen presenting cells; these could e.g. be surface molecules selected from CLEC9A and DEC205, meaning that the antibody binding region could be anti-CLEC9A, anti-DEC205, or variants thereof, such as anti-CLEC9A Fv, anti-DEC205 Fv.

Also, the APC targeting unit (iii) may consist of or comprises a ligand, such as CLEC9 peptide ligand.

The APC targeting unit (iii) is in certain embodiments selected from Xcl1, GM-CSF, anti-DEC-205 Fv, anti-CLEC9 Fv, and CLEC9 ligand.

In yet other embodiments, the APC targeting unit (iii) is a cytokine, such as GM-CSF, or another APC targeting unit (iii) that binds CD116.

In addition to or instead of the APC targeting unit the fusion polypeptide of the 1^{st} aspect of the invention or herein-described embodiments thereof may further comprise at least one amino acid sequence acting as a multimerization domain—the multimerization domain is generally referred to via the roman numeral iv herein.

It is preferred that the multimerization domain (iv) contributes to multimerization between copies of the fusion polypeptide through the formation of an interchain covalent bond, such as a disulphide bridge.

For instance, multimerization domain (iv) is or comprises a hinge region, such as an Ig hinge region, preferably an IgG-derived hinge region.

Also the multimerization domain (iv) in addition or as an alternative comprises a carboxyterminal C domain (CH3 domain), such as the carboxyterminal C domain of Ig (C_{γ}3 domain), or a sequence that is substantially homologous to said C domain, such as the CH3 domain of IgG3.

In the event the multimerization domain comprises both the hinge region and CH3 domains, these are preferably are connected by a linker sequence of amino acids, such as GGGSS (SEQ ID NO: 21), such as in triplicate sequence of the amino acids GGGSS (GGGSSGGGSSGGGSS, SEQ ID NO: 22).

In addition, or as an alternative, the multimerization domain (iv) may comprise a dimerization motif or any other multimerization motif, which participates in the multimerization through hydrophobic interactions, such as through a CH3 domain.

In some embodiments the hinge region comprises h1+h4 or h4 derived from IgG, such as an IgG2 or IgG3.

In general, the multimerization domain (iv) may in practice act as a linker, preferably between the B-cell epitope-rich region and the T-cell epitope-rich region.

In important embodiments of the fusion polypeptide of the 1^{st} aspect the B-cell epitope-rich region (i) and the T-cell epitope rich region (ii), and if relevant, the APC targeting unit (iii), and the multimerization domain (iv) are joined in any order and optionally separated by linking amino acid sequences (L).

Consequently, the fusion polypeptide may have a linear structure in the N→C direction selected from:
i-L-ii,
ii-L-i,
i-L-ii-L-iii,
ii-L-i-L-iii,
i-L-iii-L-ii,
ii-L-iii-L-i,
iii-L-i-L-ii,
iii-L-ii-L-i,
i-L-ii-L-iv,
ii-L-i-L-iv,
i-L-iv-L-ii,
ii-L-iv-L-i,
iv-L-i-L-ii,
iv-L-ii-L-i,
i-L-ii-L-iii-L-iv,
i-L-ii-L-iv-L-iii,
i-L-iii-L-i-L-iv,
i-L-iii-L-iv-L-ii,
i-L-iv-L-ii-L-iii,
i-L-iv-L-iii-L-ii,
ii-L-i-L-iii-L-iv,
ii-L-i-L-iv-L-iii,
ii-L-iii-L-i-L-iv,
ii-L-iii-L-iv-L-i,
ii-L-iv-L-i-L-iii,
ii-L-iv-L-iii-L-i,
iii-L-i-L-ii-L-iv,
iii-L-i-L-iv-L-ii,
iii-L-ii-L-i-L-iv,
iii-L-ii-L-iv-L-i,
iii-L-iv-L-i-L-ii,
iii-L-iv-L-ii-L-i,
iv-L-i-L-ii-L-iii,
iv-L-i-L-ii-L-iii,
iv-L-ii-L-i-L-iii,
iv-L-ii-L-iii-L-i,
iv-L-iii-L-i-L-ii, and
iv-L-iii-L-ii-L-i, wherein i, ii, iii, and iv refer to the B-cell epitope-rich region, the T-cell epitope-rich region, the APC targeting unit, and the multimerization domain, respectively, in line with the above, and L within each fusion polypeptide may be identical or non-identical and in each case designates a bond or a peptide linker.

The length of the linking amino acid sequences L between epitopes may—if they are present—vary. If possible, the presence of linking amino acid sequences between epitopes may be altogether avoided, e.g. if pad regions between the epitopes in their own right ensure satisfactory processing of the epitopes. On the other hand, if linking amino acid sequences are indeed present between epitopes, they typically have lengths of 3, 4, 5, 6, 7, 8, 9, 10, 11, 12, 13, 14, 15, 16, 17, 18, 19, or 20 amino acid residues, with lengths ≤10 amino acid residues being preferred.

It will be understood that the linker sequences can be regarded as separate features of the polypeptides, but depending on the exact composition of i, ii, iii, and iv, the linking sequences may—if at all present—in reality be composed of terminal amino acids from one or both the joined peptides of one of the formulae i, ii, iii, and iv. The important feature is that each of the peptides i, ii, iii, and iv are properly separated in order for them to be functional, i.e. that the presence of a neighbouring peptide does not impose a steric hindrance of the functionality of the polypeptide.

In order to increase the chances that a fusion polypeptide of the present invention induces antibodies against a relevant number of serotypes, strains, or other variants of the selected intracellular pathogen, the B-cell epitope-rich region preferably comprises non-identical fragments derived from at least two non-identical sequence variants of said at least one surface exposed protein, wherein the at least 2 non-identical fragments optionally are separated with (an) amino acid linker sequence(s).

Typically, the B-cell epitope-rich region can further comprise multiple relevant surface exposed B-cell epitope fragments found in different strains or serotypes of said pathogen to increase the efficacy of the vaccine against additional non-identical sequence variants.

Typically, the B-cell epitope-rich region further comprises CD4⁺ epitopes (T-helper epitopes) of different variants of the surface-exposed protein found in different strains or serotypes of said pathogen, and/or comprises CD4⁺ epitopes not found in said pathogen. As pointed out above, the presence of T-helper epitopes facilitates that the induced immune response involves stimulation and proliferation of B-lymphocytes and thereby production of antigen-specific antibodies. Also, the B-cell epitope rich region may in addition comprise CD8⁺ epitopes (CTL epitopes) of different variants of the surface-exposed protein found in different strains or serotypes of said pathogen.

In preferred embodiments, these CD4⁺ and/or CD8⁺ epitopes comprised in the B-cell epitope rich region are located to not disturb or to minimally change the 3-dimensional structure of the B-cell epitopes. This non-destructive positioning of the CD4⁺ and/or CD8⁺ epitopes can be attained in a number of ways: A strategy for this purpose relies on epitope grafting, i.e. a process of engineering the sequence of the B-cell epitope rich region by replacing stretches of amino acids (e.g. in the sequence of a native protein or surface-exposed fragment thereof) with peptides that are predicted to be CD4⁺ and/or CD8⁺ epitopes. The different grafting approaches can employ different means of identifying potential graft candidates from a pool of predicted epitopes (termed graft candidates). These candidates can be known CD4⁺ and/or CD8⁺ epitopes from the pathogen or sequences predicted to be CD4⁺ and/or CD8⁺ epitopes of the pathogen, or they may even be CD4⁺ and/or CD8⁺ epitopes from other sources. The grafting of such CD4⁺ and/or CD8⁺ epitopes into the sequence can be done by comparing the sequence of the CD4⁺ and/or CD8⁺ epitopes to the wildtype sequence of the spike protein. The first grafting approach relied on sequence identity, only performing grafts if the graft candidate could be aligned to the wildtype sequence with a maximum of two mismatches in the amino acid sequence (multiple sequence alignment; MSA). The second grafting approach, which is a preferred embodiment of the present design strategies, utilizes a deep neural network latent variable model to perform grafts. A useful model employs a deep Markov model (“BIFROST”) trained to generate local protein structure (Thygesen, Christian B., et al. “Efficient Generative Modelling of Protein Structure Fragments using a Deep Markov ModeL” International Conference on Machine Learning. PMLR, 2021) by encoding the sequences of graft candidates as well as the wildtype sequence into distributions over latent variables. Grafting is performed if KL-divergence between the obtained distributions is less than 13. A third grafting approach is a combination of the first two, wherein grafts are performed using the MSA approach, and then evaluated by BIFROST (MSA+BIFROST). If BIFROST identified grafts with KL-divergence over 13, the sequence is reverted to the wildtype.

The T-cell epitope-rich region (ii) of the fusion polypeptide of the 1^{st} aspect of the invention may also comprise T-cell epitopes from other proteins of the pathogen than the surface exposed protein relevant of the B-cell epitope-rich region; these T-cell epitopes serve to induce cellular immune responses against proteins of the intracellular pathogen, and not necessarily immune responses that are directed towards protein which is exposed to the extracellular phase. For instance, proteins that are responsible for the intracellular activity of virus (e.g. enzymes involved in replication, transcription or translation of the genetic material in a virus) may serve as excellent targets for cellular immunity, but are largely irrelevant as targets for a humeral immune response.

In preferred embodiments, the T-cell epitope region comprises T-cell epitopes and/or T-cell epitope hotspots derived from at least 2 different strains or serotypes of said pathogen; the number of different strains and serotypes from which T-cell epitopes or T-cell epitope hotspots are derived will depend on the intracellular pathogen—in some cases, only a few serotypes are relevant as pathogens in humans, thus limiting the number of necessary sources for T-cell epitopes and T-cell epitope hotspots, in other cases a substantial number of serotypes are relevant, thereby increasing the need for many sources.

It may be convenient that T-cell epitopes and/or T-cell epitope hotspots of the T-cell epitope rich region are separated by linking amino acid sequences. In addition, the T-cell epitope-rich region may comprise amino acid sequences (pad regions), which are amino acid sequences that facilitate correct antigen processing and antigen presentation of T-cell epitopes. A pad region is typically 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues in length.

Selection of T-cell epitope hotspots from the intracellular pathogen will typically rely on the density of T-cell epitopes. The higher the number of T-cell epitopes per amino acid for an HLA allele, the more relevant the hotspot is. When investigating the proteinaceous expression products from the pathogen, for instance by examining the sequence information in the transcribed genes of the pathogen, for the presence of encoded HLA ligands (preferably those which are also T-cell receptor binders in humans), the number of HLA ligands/T-cell epitopes will naturally vary along the amino acid sequence of the expression products, and for each pathogen there will exist an average number of T-cell epitopes per amino acid. T-cell epitope hotspots are hence selected from those stretches of amino acid residues in the expression products where the density of epitopes is above this average, normally considerable above this average. However, the selection of T-cell epitope rich regions is not the only criterion for deciding on a hotspot to include in the constructs of the invention: For the final vaccine, it is of importance to ensure that immunity is induced in as large a fraction of the population as possible, which means that the hotspots included in combination will provide for a maximized population coverage; this is ensured by selecting the combination of regions which provide for ligands to as many MHC molecules as possible, while at the same time ensuring that these ligands together match the largest population coverage. It is also necessary to ensure that both MHC class I and II ligands are sufficiently represented. To give a simplified example, if selection required inclusion of 2 of 3 identified epitope rich regions, where two of the regions include a ligand for a very common HLA molecule as well as ligands for very uncommon HLA molecules, whereas the 3^{rd} includes a ligand for a “medium-common” HLA molecule, it is highly likely that optimization of population coverage would mean that the 3^{rd} region is included together with one of the 2 other, even when the epitope density in the 3^{rd} region should be lower than in the 2 other regions.

One simple way to provisionally select a hotspot region is nevertheless to determine the number of encoded T-cell epitopes per kilobase transcriptome in the organism in question. A T-cell epitope hotspot is a transcriptome region where the number of encoded T-cell epitopes per kilobase is at least twice the number of encoded T-cell epitopes per kilobase of the entire transcriptome.

Number of T-cell epitopes per amino acid for an HLA allele will preferably be at least 0.010, such as at least at least 0.011, at least 0.012, at least 0.013, at least 0.014, at least 0.015, at least 0.016, at least 0.017, at least 0.018, at least 0.019, at least 0.02, at least 0.021, at least 0.022, at least 0.023, at least 0.024, at least 0.025, at least 0.026, at least 0.027, at least 0.028, at least 0.029, at least 0.03, at least 0.031, at least 0.032, at least 0.033, at least 0.034, at least 0.035, at least 0.036, at least 0.037, at least 0.038, at least 0.039, at least 0.04, at least 0.041, at least 0.042, at least 0.043, at least 0.044, at least 0.045, at least 0.046, at least 0.047, at least 0.048, at least 0.049, at least 0.05, at least 0.051, at least 0.052, at least 0.053, at least 0.054, at least 0.055, at least 0.056, at least 0.057, at least 0.058, at least 0.059, at least 0.06, at least 0.061, at least 0.062, at least 0.063, at least 0.064, at least 0.065, at least 0.066, at least 0.067, at least 0.068, at least 0.069, at least 0.07, at least 0.071, at least 0.072, at least 0.073, at least 0.074, at least 0.075, at least 0.076, at least 0.077, at least 0.078, at least 0.079, at least 0.08, at least 0.081, at least 0.082, at least 0.083, at least 0.084, at least 0.085, at least 0.086, at least 0.087, at least 0.088, at least 0.089, at least 0.09, at least 0.091, at least 0.092, at least 0.093, at least 0.094, at least 0.095, at least 0.096, at least 0.097, at least 0.098, at least 0.099, at least 0.1, at least 0.101, at least 0.102, at least 0.103, at least 0.104, at least 0.105, at least 0.106, at least 0.107, at least 0.108, at least 0.109, at least 0.11, at least 0.111, at least 0.112, at least 0.113, at least 0.114, at least 0.115, at least 0.116, at least 0.117, at least 0.118, at least 0.119, at least 0.12, at least 0.121, at least 0.122, at least 0.123, at least 0.124, at least 0.125, at least 0.126, at least 0.127, at least 0.128, at least 0.129, at least 0.13, at least 0.131, at least 0.132, at least 0.133, at least 0.134, at least 0.135, at least 0.136, at least 0.137, at least 0.138, at least 0.139, at least 0.14, at least 0.141, at least 0.142, at least 0.143, at least 0.144, at least 0.145, at least 0.146, at least 0.147, at least 0.148, at least 0.149, at least 0.15, at least 0.151, at least 0.152, at least 0.153, at least 0.154, at least 0.155, at least 0.156, at least 0.157, at least 0.158, at least 0.159, at least 0.16, at least 0.161, at least 0.162, at least 0.163, at least 0.164, at least 0.165, at least 0.166, at least 0.167, at least 0.168, at least 0.169, at least 0.17, at least 0.171, at least 0.172, at least 0.173, at least 0.174, at least 0.175, at least 0.176, at least 0.177, at least 0.178, at least 0.179, at least 0.18, at least 0.181, at least 0.182, at least 0.183, at least 0.184, at least 0.185, at least 0.186, at least 0.187, at least 0.188, at least 0.189, at least 0.19, at least 0.191, at least 0.192, at least 0.193, at least 0.194, at least 0.195, at least 0.196, at least 0.197, at least 0.198, at least 0.199, at least 0.2, at least 0.201, at least 0.202, at least 0.203, at least 0.204, at least 0.205, at least 0.206, at least 0.207, at least 0.208, at least 0.209, at least 0.21, at least 0.211, at least 0.212, at least 0.213, at least 0.214, at least 0.215, at least 0.216, at least 0.217, at least 0.218, at least 0.219, at least 0.22, at least 0.221, at least 0.222, at least 0.223, at least 0.224, at least 0.225, at least 0.226, at least 0.227, at least 0.228, at least 0.229, at least 0.23, at least 0.231, at least 0.232, at least 0.233, at least 0.234, at least 0.235, at least 0.236, at least 0.237, at least 0.238, at least 0.239, at least 0.24, at least 0.241, at least 0.242, at least 0.243, at least 0.244, at least 0.245, at least 0.246, at least 0.247, at least 0.248, at least 0.249, at least 0.25, at least 0.251, at least 0.252, at least 0.253, at least 0.254, at least 0.255, at least 0.256, at least 0.257, at least 0.258, at least 0.259, at least 0.26, at least 0.261, at least 0.262, at least 0.263, at least 0.264, at least 0.265, at least 0.266, at least 0.267, at least 0.268, at least 0.269, at least 0.27, at least 0.271, at least 0.272, at least 0.273, at least 0.274, at least 0.275, at least 0.276, at least 0.277, at least 0.278, at least 0.279, at least 0.28, at least 0.281, at least 0.282, at least 0.283, at least 0.284, at least 0.285, at least 0.286, at least 0.287, at least 0.288, at least 0.289, at least 0.29, at least 0.291, at least 0.292, at least 0.293, at least 0.294, at least 0.295, at least 0.296, at least 0.297, at least 0.298, at least 0.299, at least 0.3, at least 0.301, at least 0.302, at least 0.303, at least 0.304, at least 0.305, at least 0.306, at least 0.307, at least 0.308, at least 0.309, at least 0.31, at least 0.311, at least 0.312, at least 0.313, at least 0.314, at least 0.315, at least 0.316, at least 0.317, at least 0.318, at least 0.319, at least 0.32, at least 0.321, at least 0.322, at least 0.323, at least 0.324, at least 0.325, at least 0.326, at least 0.327, at least 0.328, at least 0.329, at least 0.33, at least 0.331, at least 0.332, at least 0.333, at least 0.334, at least 0.335, at least 0.336, at least 0.337, at least 0.338, at least 0.339, at least 0.34, at least 0.341, at least 0.342, at least 0.343, at least 0.344, at least 0.345, at least 0.346, at least 0.347, at least 0.348, at least 0.349, at least 0.35, at least 0.351, at least 0.352, at least 0.353, at least 0.354, at least 0.355, at least 0.356, at least 0.357, at least 0.358, at least 0.359, at least 0.36, at least 0.361, at least 0.362, at least 0.363, at least 0.364, at least 0.365, at least 0.366, at least 0.367, at least 0.368, at least 0.369, at least 0.37, at least 0.371, at least 0.372, at least 0.373, at least 0.374, at least 0.375, at least 0.376, at least 0.377, at least 0.378, at least 0.379, at least 0.38, at least 0.381, at least 0.382, at least 0.383, at least 0.384, at least 0.385, at least 0.386, at least 0.387, at least 0.388, at least 0.389, at least 0.39, at least 0.391, at least 0.392, at least 0.393, at least 0.394, at least 0.395, at least 0.396, at least 0.397, at least 0.398, at least 0.399, at least 0.4, at least 0.401, at least 0.402, at least 0.403, at least 0.404, at least 0.405, at least 0.406, at least 0.407, at least 0.408, at least 0.409, at least 0.41, at least 0.411, at least 0.412, at least 0.413, at least 0.414, at least 0.415, at least 0.416, at least 0.417, at least 0.418, at least 0.419, at least 0.42, at least 0.421, at least 0.422, at least 0.423, at least 0.424, at least 0.425, at least 0.426, at least 0.427, at least 0.428, at least 0.429, at least 0.43, at least 0.431, at least 0.432, at least 0.433, at least 0.434, at least 0.435, at least 0.436, at least 0.437, at least 0.438, at least 0.439, at least 0.44, at least 0.441, at least 0.442, at least 0.443, at least 0.444, at least 0.445, at least 0.446, at least 0.447, at least 0.448, at least 0.449, at least 0.45, at least 0.451, at least 0.452, at least 0.453, at least 0.454, at least 0.455, at least 0.456, at least 0.457, at least 0.458, at least 0.459, at least 0.46, at least 0.461, at least 0.462, at least 0.463, at least 0.464, at least 0.465, at least 0.466, at least 0.467, at least 0.468, at least 0.469, at least 0.47, at least 0.471, at least 0.472, at least 0.473, at least 0.474, at least 0.475, at least 0.476, at least 0.477, at least 0.478, at least 0.479, at least 0.48, at least 0.481, at least 0.482, at least 0.483, at least 0.484, at least 0.485, at least 0.486, at least 0.487, at least 0.488, at least 0.489, at least 0.49, at least 0.491, at least 0.492, at least 0.493, at least 0.494, at least 0.495, at least 0.496, at least 0.497, at least 0.498, at least 0.499, at least 0.5, at least 0.501, at least 0.502, at least 0.503, at least 0.504, at least 0.505, at least 0.506, at least 0.507, at least 0.508, at least 0.509, at least 0.51, at least 0.511, at least 0.512, at least 0.513, at least 0.514, at least 0.515, at least 0.516, at least 0.517, at least 0.518, at least 0.519, at least 0.52, at least 0.521, at least 0.522, at least 0.523, at least 0.524, at least 0.525, at least 0.526, at least 0.527, at least 0.528, at least 0.529, at least 0.53, at least 0.531, at least 0.532, at least 0.533, at least 0.534, at least 0.535, at least 0.536, at least 0.537, at least 0.538, at least 0.539, at least 0.54, at least 0.541, at least 0.542, at least 0.543, at least 0.544, at least 0.545, at least 0.546, at least 0.547, at least 0.548, at least 0.549, at least 0.55, at least 0.551, at least 0.552, at least 0.553, at least 0.554, at least 0.555, at least 0.556, at least 0.557, at least 0.558, at least 0.559, at least 0.56, at least 0.561, at least 0.562, at least 0.563, at least 0.564, at least 0.565, at least 0.566, at least 0.567, at least 0.568, at least 0.569, at least 0.57, at least 0.571, at least 0.572, at least 0.573, at least 0.574, at least 0.575, at least 0.576, at least 0.577, at least 0.578, at least 0.579, at least 0.58, at least 0.581, at least 0.582, at least 0.583, at least 0.584, at least 0.585, at least 0.586, at least 0.587, at least 0.588, at least 0.589, at least 0.59, at least 0.591, at least 0.592, at least 0.593, at least 0.594, at least 0.595, at least 0.596, at least 0.597, at least 0.598, at least 0.599, at least 0.6, at least 0.601, at least 0.602, at least 0.603, at least 0.604, at least 0.605, at least 0.606, at least 0.607, at least 0.608, at least 0.609, at least 0.61, at least 0.611, at least 0.612, at least 0.613, at least 0.614, at least 0.615, at least 0.616, at least 0.617, at least 0.618, at least 0.619, at least 0.62, at least 0.621, at least 0.622, at least 0.623, at least 0.624, at least 0.625, at least 0.626, at least 0.627, at least 0.628, at least 0.629, at least 0.63, at least 0.631, at least 0.632, at least 0.633, at least 0.634, at least 0.635, at least 0.636, at least 0.637, at least 0.638, at least 0.639, at least 0.64, at least 0.641, at least 0.642, at least 0.643, at least 0.644, at least 0.645, at least 0.646, at least 0.647, at least 0.648, at least 0.649, at least 0.65, at least 0.651, at least 0.652, at least 0.653, at least 0.654, at least 0.655, at least 0.656, at least 0.657, at least 0.658, at least 0.659, at least 0.66, at least 0.661, at least 0.662, at least 0.663, at least 0.664, at least 0.665, at least 0.666, at least 0.667, at least 0.668, at least 0.669, at least 0.67, at least 0.671, at least 0.672, at least 0.673, at least 0.674, at least 0.675, at least 0.676, at least 0.677, at least 0.678, at least 0.679, at least 0.68, at least 0.681, at least 0.682, at least 0.683, at least 0.684, at least 0.685, at least 0.686, at least 0.687, at least 0.688, at least 0.689, at least 0.69, at least 0.691, at least 0.692, at least 0.693, at least 0.694, at least 0.695, at least 0.696, at least 0.697, at least 0.698, at least 0.699, at least 0.7, at least 0.701, at least 0.702, at least 0.703, at least 0.704, at least 0.705, at least 0.706, at least 0.707, at least 0.708, at least 0.709, at least 0.71, at least 0.711, at least 0.712, at least 0.713, at least 0.714, at least 0.715, at least 0.716, at least 0.717, at least 0.718, at least 0.719, at least 0.72, at least 0.721, at least 0.722, at least 0.723, at least 0.724, at least 0.725, at least 0.726, at least 0.727, at least 0.728, at least 0.729, at least 0.73, at least 0.731, at least 0.732, at least 0.733, at least 0.734, at least 0.735, at least 0.736, at least 0.737, at least 0.738, at least 0.739, at least 0.74, at least 0.741, at least 0.742, at least 0.743, at least 0.744, at least 0.745, at least 0.746, at least 0.747, at least 0.748, at least 0.749, at least 0.75, at least 0.751, at least 0.752, at least 0.753, at least 0.754, at least 0.755, at least 0.756, at least 0.757, at least 0.758, at least 0.759, at least 0.76, at least 0.761, at least 0.762, at least 0.763, at least 0.764, at least 0.765, at least 0.766, at least 0.767, at least 0.768, at least 0.769, at least 0.77, at least 0.771, at least 0.772, at least 0.773, at least 0.774, at least 0.775, at least 0.776, at least 0.777, at least 0.778, at least 0.779, at least 0.78, at least 0.781, at least 0.782, at least 0.783, at least 0.784, at least 0.785, at least 0.786, at least 0.787, at least 0.788, at least 0.789, at least 0.79, at least 0.791, at least 0.792, at least 0.793, at least 0.794, at least 0.795, at least 0.796, at least 0.797, at least 0.798, at least 0.799, at least 0.8, at least 0.801, at least 0.802, at least 0.803, at least 0.804, at least 0.805, at least 0.806, at least 0.807, at least 0.808, at least 0.809, at least 0.81, at least 0.811, at least 0.812, at least 0.813, at least 0.814, at least 0.815, at least 0.816, at least 0.817, at least 0.818, at least 0.819, at least 0.82, at least 0.821, at least 0.822, at least 0.823, at least 0.824, at least 0.825, at least 0.826, at least 0.827, at least 0.828, at least 0.829, at least 0.83, at least 0.831, at least 0.832, at least 0.833, at least 0.834, at least 0.835, at least 0.836, at least 0.837, at least 0.838, at least 0.839, at least 0.84, at least 0.841, at least 0.842, at least 0.843, at least 0.844, at least 0.845, at least 0.846, at least 0.847, at least 0.848, at least 0.849, at least 0.85, at least 0.851, at least 0.852, at least 0.853, at least 0.854, at least 0.855, at least 0.856, at least 0.857, at least 0.858, at least 0.859, at least 0.86, at least 0.861, at least 0.862, at least 0.863, at least 0.864, at least 0.865, at least 0.866, at least 0.867, at least 0.868, at least 0.869, at least 0.87, at least 0.871, at least 0.872, at least 0.873, at least 0.874, at least 0.875, at least 0.876, at least 0.877, at least 0.878, at least 0.879, at least 0.88, at least 0.881, at least 0.882, at least 0.883, at least 0.884, at least 0.885, at least 0.886, at least 0.887, at least 0.888, at least 0.889, at least 0.89, at least 0.891, at least 0.892, at least 0.893, at least 0.894, at least 0.895, at least 0.896, at least 0.897, at least 0.898, at least 0.899, at least 0.9, at least 0.901, at least 0.902, at least 0.903, at least 0.904, at least 0.905, at least 0.906, at least 0.907, at least 0.908, at least 0.909, at least 0.91, at least 0.911, at least 0.912, at least 0.913, at least 0.914, at least 0.915, at least 0.916, at least 0.917, at least 0.918, at least 0.919, at least 0.92, at least 0.921, at least 0.922, at least 0.923, at least 0.924, at least 0.925, at least 0.926, at least 0.927, at least 0.928, at least 0.929, at least 0.93, at least 0.931, at least 0.932, at least 0.933, at least 0.934, at least 0.935, at least 0.936, at least 0.937, at least 0.938, at least 0.939, at least 0.94, at least 0.941, at least 0.942, at least 0.943, at least 0.944, at least 0.945, at least 0.946, at least 0.947, at least 0.948, at least 0.949, at least 0.95, at least 0.951, at least 0.952, at least 0.953, at least 0.954, at least 0.955, at least 0.956, at least 0.957, at least 0.958, at least 0.959, at least 0.96, at least 0.961, at least 0.962, at least 0.963, at least 0.964, at least 0.965, at least 0.966, at least 0.967, at least 0.968, at least 0.969, at least 0.97, at least 0.971, at least 0.972, at least 0.973, at least 0.974, at least 0.975, at least 0.976, at least 0.977, at least 0.978, at least 0.979, at least 0.98, at least 0.981, at least 0.982, at least 0.983, at least 0.984, at least 0.985, at least 0.986, at least 0.987, at least 0.988, at least 0.989, at least 0.99, at least 0.991, at least 0.992, at least 0.993, at least 0.994, at least 0.995, at least 0.996, at least 0.997, at least 0.998, at least 0.999, and at least 1.000.

The intracellular pathogen is normally selected from the group consisting of a virus, a protozoan, a bacterium, and a fungus. Where all virus by nature are intracellular pathogens, this is not the case for the other pathogens of this list. A few bacteria are known to be almost exclusively intracellular (e.g. M. tuberculosis), where others are capable of establishing an intracellular presence (e.g. S. typhi). Irrespective of this, the presently disclosed fusion polypeptides are useful in combatting or preventing diseases caused by these intracellular pathogens.

In important embodiments the intracellular pathogen is a virus, and in that cases it will often be selected from the groups of Arenavirus, Herpesvirus, Poxvirus, Asfarviridae, Flavivirus, Alphavirus, Togavirus, Coronavirus, Hepatitis virus (A, B, C, D, or E), Orthomyxovirus, Paramyxovirus, Rhabdovirus, Bunyavirus, Filovirus, Retrovirus, Poxvirus, Adenovirus, Papillomavirus, Reovirus, Picornavirus, Calicivirus, and Astrovirus. Particular relevant virus are SARS-Cov 1, SARS-Cov 2, MERS-COV, HCoV-229E, HCoV-NL63, HCoV-OC43, HCoV-HKU1 HIV (1 or 2), influenza virus (A, B, or C), Ebola virus, RSV, and Lassa virus.

When the intracellular pathogen is a virus, the at least one surface exposed protein is preferably a membrane fusion protein (MFP, i.e. a protein responsible for effecting fusion of the virus envelope with the cell membrane) preferably of the type I or a receptor binding domain. Such MFP is preferably selected from a spike protein from coronavirus (including any of the coronavirus discussed above), in particular from SARS-Cov 2, a hemagglutinin, in particular an influenza virus hemagglutinin, Ebola Glycoprotein (GP), Lassa Glycoprotein, HIV-1 Envelope, or an RSV Fusion glycol protein (RSV-F).

In the event the intracellular pathogen is a bacterium, it will typically be selected from
a mycobacterium species, such as M. tuberculosis, M. leprae, and M. lepromatosis;
a Salmonella species, such as S. typhi;
a Rickettsia species, such as R. akari, R. rickettsia, R. conori, R. australis, R. felis, R. japonica, and R. africae;
a Chlamydia species, such as C. trachomatis, C. abortus, C. psittaci, and C. pneumoniae; Coxiella burneti;
Bartonella henselae;
Francisella tularensi;
Listeria monocytogenes;
a Brucella species;
a Legionella species, such as L. pneumophila;
a Nocardia species,
a Neisseria species, such as N. gonorrhoea;
a Yersinia species, such as Y. pestis and Y. enterocolitica;
Shigella flexneri; and
Staphylococcus aureus.

In the event the intracellular pathogen is a protozoa, it will typically be selected from
a Plasmodium species, such as P. falciparum, P. vivax, P. malariae, P. ovale, and P. knowlesi;
a Toxoplasma species, such as T. gondii;
a Cryptosporidium species, such as C. parvum;
a Leishmania species; and
Trypanosoma cruzi.

Finally, when the intracellular pathogen is a fungus, it will typically be selected from a Pneumocystis species, in particular P. jirovecii (previously P. carinii).

### 2^{nd} Aspect of the Invention

The nucleic acid fragment of the 2^{nd} aspect of the invention is typically a DNA or RNA fragment—both are useful as therapeutic agents per se (in DNA or RNA vaccination), but are also important tools in recombinant preparation of the fusion polypeptide of the first aspect of the invention.

The nucleic acid fragment of the 2^{nd} aspect of the invention, option b, will typically encode a polypeptide wherein the B-cell epitope region (i) is fused N- or C-terminally, directly or via a linking amino acid sequence, to a multimerization domain (iv) as described above in embodiments of the 1^{st} aspect of the invention and/or wherein the T-cell epitope-rich region (ii) is fused N- or C-terminally, directly or via a linking amino acid sequence, to a multimerization domain (iv) as described in embodiments of the 1^{st} aspect of the invention.

Further, the nucleic acid fragment as described in option b, will preferably encode A) the T-cell epitope-rich region fused N- or C-terminally, directly or via a linking amino acid sequence, to an APC targeting unit (iii), or B) the B-cell epitope-rich region fused N- or C-terminally, directly or via a linking amino acid sequence, to an APC targeting unit (iii), or C) the B-cell epitope-rich region and the T-cell epitope-rich regions each fused N- or C-terminally, directly or via a linking amino acid sequence, to an APC targeting unit (iii), wherein said APC targeting unit (iii) is described above in embodiments of the 1^{st} aspect of the invention.

Typically, and for use in both therapy and as a production tool, the nucleic acid fragment of the second aspect and embodiments disclosed herein of the 2^{nd} aspect, is under the control of a promoter. If several polypeptides are encoded by the nucleic acid fragment, the nucleic acid sequence encoding each polypeptide can be under the control of separate promoters.

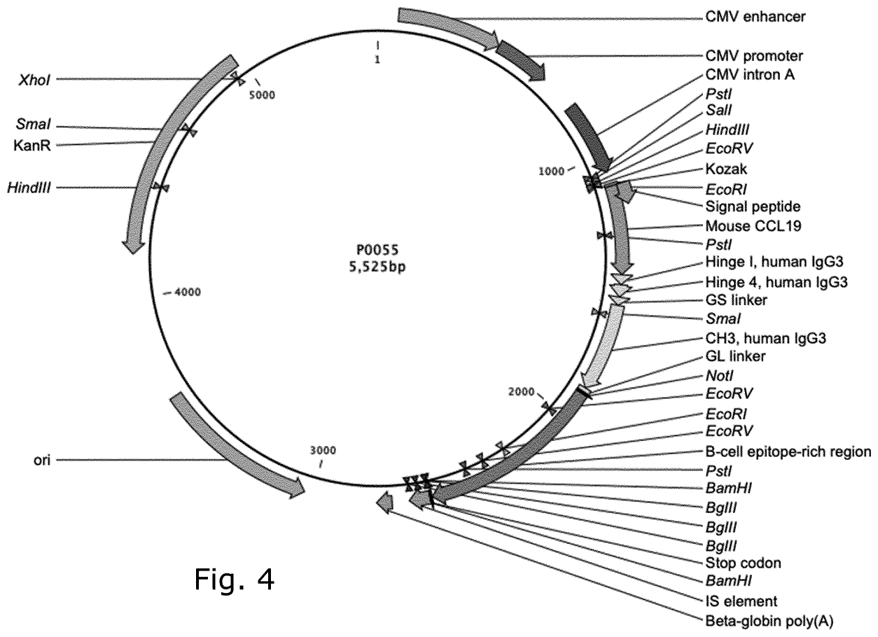

7 specific nucleic acid constructs that encode fusion polypeptides of the invention have been designed by now (FIG. 4). These specific constructs are designed to target SARS-Cov-2 (the causative agent of COVID-19).

In the 5′-3′ direction, these constructs have the general structures set forth in in FIG. 3 or 4.

### 3^{rd} Aspect of the Invention

Expression vectors of the invention find use in recombinant production of the fusion polypeptides of the 1^{st} aspect of the invention but also as therapeutic agents.

Expression vectors of the invention fall into several categories discussed infra. One preferred vector of the invention comprises in operable linkage and in the 5′-3′ direction, an expression control region comprising an enhancer/promoter for driving expression of the nucleic acid fragment defined for option i) above, optionally a signal peptide coding sequence, a nucleotide sequence defined for option i), and optionally a terminator. Hence, such a vector constitutes an expression vector useful for effecting production in cells of the fusion polypeptide of the 1^{st} aspect of the invention. Recombinant production is conveniently effected in bacterial host cells, so here it is preferred that the expression control region drives expression in prokaryotic cell such as a bacterium, e.g. in E coli. However, if the vector is to drive expression in mammalian cell (as would be the case for a DNA or RNA vaccine vector), the expression control region should be adapted to this particular use.

The vector may further comprise a sequence encoding a signal peptide, which may provide for secretion or membrane integration of the expression product from said vector. For the purposes of nucleic acid vaccination, the signal peptides encoded are typically selected from those described in Williams J. A. Vaccines (Basel). 2013 September; 1(3): 225-249 as well as in the references cited therein.

For DNA expression vectors, such as plasmid expression vectors, an interesting option is to include somewhere in the vector, but typically outside the region that encodes the fusion polypeptide of the invention, a nucleotide sequence, which facilitates import into the nucleus of the expression vector. Such nucleotide sequences are well-known in the art and are typically short consensus motifs recognized by specific transcription factors. They can be used to actively shuttle, upon activation and following an importin-mediated process, plasmid from the cytosol to the nucleus through nuclear pores. Cf. van Gaal EVB et al., Pharm Res. 2011, 28(7):1707-172.

At any rate, certain vectors of the invention are capable of autonomous replication.

Also, the vector of the invention may be one that is capable of being integrated into the genome of a host cell—this is particularly useful if the vector is use in the production of stably transformed cells, where the progeny will also include the genetic information introduced via the vector. Alternatively, vectors incapable of being integrated into the genome of a mammalian host cell are useful in e.g. nucleic acid vaccination.

Typically, the expression vector of the invention is selected from the group consisting of a virus, such as a attenuated virus (which may in itself be useful as a vaccine agent), a bacteriophage, a plasmid, a mini plasmid, a minichromosome, and a cosmid. For all expression vectors that are intended for administration as a drug, the vector should be pharmaceutically acceptable and hence non-pathogenic.

A more detailed discussion of expression vectors useful in the invention is provided in the following:

Fusion polypeptides of the invention may be encoded by a nucleic acid molecule comprised in a vector. A nucleic acid sequence can be “heterologous,” which means that it is in a context foreign to the cell in which the vector is being introduced, which includes a sequence homologous to a sequence in the cell but in a position within the host cell where it is ordinarily not found. Vectors include naked DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example the handbooks by Sambrook et al, 2001; Ausubel et al, 1996, both incorporated herein by reference). In addition to encoding the fusion polypeptides of this invention, a vector of the present invention may encode polypeptide sequences such as a tag or immunogenicity enhancing peptide (e.g. an immunogenic carrier or a fusion partner that stimulates the immune system, such as a cytokine or active fragment thereof). Useful vectors encoding such fusion proteins include pIN vectors, vectors encoding a stretch of histidines, and pGEX vectors, for use in generating glutathione S-transferase (GST) soluble fusion proteins for later purification and separation or cleavage.

Expression vectors of the invention may be used in a host cell to produce a polypeptide of the invention that may subsequently be purified for administration to a subject or the vector may be purified for direct administration to a subject for expression of the protein in the subject (as is the case when administering a nucleic acid vaccine).

Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

Numerous plasmids known in the art may be used for the production of nucleic acid vaccines. Suitable embodiments of the nucleic acid vaccine employ constructs using the plasmids VR1012 (Vical Inc., San Diego Calif.), pCMVI.UBF3/2 (S. Johnston, University of Texas), pTVG4 (Johnson et al., 2006, Vaccine 24(3); 293-303), pVAX1 (Thermo Fisher Scientific), or pcDNA3.1 (InVitrogen Corporation, Carlsbad, Calif.) as the vector.

Also, the pVax1 μlasmid utilised in WO 2020/182901 is an interesting vector for use in the present invention. Further, the use of immunostimulatory sequences (ISS) in the plasmid vector, such as pTVG4, as disclosed in WO 2020/182901 is also an interesting aspect of the present invention.

### 1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

Naturally, it may be important to employ a promoter and/or enhancer that effectively direct(s) the expression of the DNA segment in the cell type or organism chosen for expression. Those of skill in the art of molecular biology generally know the use of promoters, enhancers, and cell type combinations for protein expression (see Sambrook et al, 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Examples of inducible elements, which are regions of a nucleic acid sequence that can be activated in response to a specific stimulus, include but are not limited to Immunoglobulin Heavy Chain, Immunoglobulin Light Chain, T Cell Receptor, HLA DQα and/or DQβ, β-Interferon, Interleukin-2, Interleukin-2 Receptor, MHC Class II 5, MHC Class II HLA-DRa, β-Actin, Muscle Creatine Kinase (MCK), Prealbumin (Transthyretin), Elastase I, Metallothionein (MTII), Collagenase, Albumin, α-Fetoprotein, γ-Globin, β-Globin, c-fos, c-HA-ras, Insulin, Neural Cell Adhesion Molecule (NCAM), al-Antitrypain, H2B (TH2B) Histone, Mouse and/or Type I Collagen, Glucose-Regulated Proteins (GRP94 and GRP78), Rat Growth Hormone, Human Serum Amyloid A (SAA), Troponin I (TN I), Platelet-Derived Growth Factor (PDGF), Duchenne Muscular Dystrophy, SV40, Polyoma, Retroviruses, Papilloma Virus, Hepatitis B Virus, Human Immunodeficiency Virus, Cytomegalovirus (CMV) IE, and Gibbon Ape Leukemia Virus.

Inducible Elements include MT II—Phorbol Ester (TFA)/Heavy metals; MMTV (mouse mammary tumor virus)—Glucocorticoids; β-Interferon—poly(rl)×/poly(rc); Adenovirus 5 E2—EIA; Collagenase—Phorbol Ester (TPA); Stromelysin—Phorbol Ester (TPA); SV40—Phorbol Ester (TPA); Murine MX Gene—Interferon, Newcastle Disease Virus; GRP78 Gene—A23187; α-2-Macroglobulin—IL-6; Vimentin—Serum; MHC Class I Gene H-2Kb—Interferon; HSP70—E1A/SV40 Large T Antigen; Proliferin—Phorbol Ester/TPA; Tumor Necrosis Factor—PMA; and Thyroid Stimulating Hormonea Gene—Thyroid Hormone.

Also contemplated as useful in the present invention are the dectin-1 and dectin-2 promoters. Additionally, any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

The particular promoter that is employed to control the expression of peptide or protein encoding polynucleotide of the invention is not believed to be critical, so long as it is capable of expressing the polynucleotide in a targeted cell, preferably a human cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

In various embodiments, the human cytomegalovirus (CMV) immediate early gene promoter, the SV40 early promoter, and the Rous sarcoma virus long terminal repeat can be used to obtain high level expression of a related polynucleotide to this invention. The use of other viral or mammalian cellular or bacterial phage promoters, which are well known in the art, to achieve expression of polynucleotides is contemplated as well.

In embodiments in which a vector is administered to a subject for expression of the protein, it is contemplated that a desirable promoter for use with the vector is one that is not down-regulated by cytokines or one that is strong enough that even if down-regulated, it produces an effective amount of the protein/polypeptide of the current invention in a subject to elicit an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters. 2. Initiation Signals and Internal Ribosome Binding Sites (IRES) A specific initiation signal also may be required for efficient translation of coding sequences.

These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

### 2. Multiple Cloning Sites

Expression vectors can include a multiple cloning site (MCS), which is a nucleic acid region that contains multiple restriction enzyme sites, any of which can be used in conjunction with standard recombinant technology to digest the vector. Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology.

### 3. Splicing Sites

Most transcribed eukaryotic RNA molecules will undergo RNA splicing to remove introns from the primary transcripts. If relevant in the context of vectors of the present invention, vectors containing genomic eukaryotic sequences may require donor and/or acceptor splicing sites to ensure proper processing of the transcript for protein expression.

### 4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

### 5. Polyadenylation Signals

In expression, particularly eukaryotic expression (as is relevant in nucleic acid vaccination), one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

### 6. Origins of Replication

In order to propagate a vector in a host cell, it may contain one or more origins of replication sites (often termed “on”), which is a specific nucleic acid sequence at which replication is initiated. Alternatively an autonomously replicating sequence (ARS) can be employed if the host cell is yeast.

### 7. Selectable and Screenable Markers

In certain embodiments of the invention, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein of the invention. Further examples of selectable and screenable markers are well known to one of skill in the art.

4^{th}-7^{th} Aspects of the Invention

These aspects all relate to pharmaceutical compositions, i.e. vaccines or immunization compositions. Pharmaceutical compositions, in particular vaccines, according to the invention may either be prophylactic (i.e. suited to prevent infection) or therapeutic (i.e. to treat disease after infection).

In some embodiments of the invention, the pharmaceutical compositions such as vaccines include merely one single antigen, immunogen, polypeptide, protein, an in particular nucleic acid or vector of the invention, but in other embodiments, the pharmaceutical compositions comprise “cocktails” of the antigens or of the immunogens or of the polypeptides or of the protein or in particular of the nucleic acids or of the vectors of the invention.

Vaccines of the invention typically comprise immunising antigen(s), immunogen(s), polypeptide(s), protein(s) or in particular nucleic acid(s), usually in combination with “pharmaceutically acceptable carriers”, which include any carrier that does not itself induce the production of antibodies harmful to the individual receiving the composition or targeting the protein/pathogen. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immune stimulating agents (“adjuvants”). Furthermore, the antigen or immunogen may be conjugated to a bacterial toxoid, such as a toxoid from diphtheria, tetanus, cholera, H. pylori, etc. pathogen, cf. the description of immunogenic carriers supra. However, since the fusion polypeptides are derived from organisms that are regarded as foreign (non-self) by the mammalian immune system, the need for inclusion of such carriers is not of the highest relevance, as the necessary T-cell help is triggered by T-helper epitopes of the fusion polypeptide described supra.

The polypeptide-containing pharmaceutical compositions of the invention typically contain an immunological adjuvant, which is commonly an aluminium based adjuvant or one of the other adjuvants described in the following: Preferred adjuvants to enhance effectiveness of the composition include, but are not limited to: (1) aluminum salts (alum), such as aluminium hydroxide, aluminium phosphate, aluminium sulfate, etc; (2) oil-in-water emulsion formulations (with or without other specific immunostimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, MA), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi adjuvant system (RAS), (Ribi Immunochem, Hamilton, MT) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™); (3) saponin adjuvants such as Stimulon™ (Cambridge Bioscience, Worcester, MA) may be used or particles generated therefrom such as ISCOMs (immunostimulating complexes); (4) Complete Freund's Adjuvant (CFA) and Incomplete Freund's Adjuvant (IFA); (5) cytokines, such as interleukins (eg. IL-1, IL-2, IL-4, IL-5, IL-6, IL-7, IL-12, etc.), interferons (eg. gamma interferon), macrophage colony stimulating factor (M-CSF), tumor necrosis factor (TNF), etc.; and (6) other substances that act as immunostimulating agents to enhance the effectiveness of the composition. Alum and MF59™ adjuvants are preferred as antibody inducing adjuvants.

Muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

The immunogenic compositions (e.g. the immunising antigen or immunogen or polypeptide or protein or in particular nucleic acid, and the pharmaceutically acceptable carrier and/or adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic polypeptide-containing compositions used as vaccines comprise an immunologically effective amount of the antigenic or immunogenic polypeptides, as well as any other of the above-mentioned components, as needed. By “immunollogically effective amount”, it is meant that the administration of that amount to an individual, either in a single dose or as part of a series, is effective for treatment or prevention. This amount varies depending upon the health and physical condition of the individual to be treated, the taxonomic group of individuals to be treated (eg. nonhuman primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, e.g. by injection, either subcutaneously, intramuscularly, or transdermally/transcutaneously (eg. WO 98/20734). Additional formulations suitable for other modes of administration include oral, pulmonary and nasal formulations, suppositories, and transdermal applications. In the case of nucleic acid vaccination, also the intravenous or intraarterial routes may be applicable.

Dosage treatment may be a single dose schedule or a multiple dose schedule. The vaccine may be administered in conjunction with other immunoregulatory agents.

As an important alternative to protein-based vaccines, DNA vaccination (also termed nucleic acid vaccination or gene vaccination) may be used [e.g. Robinson & Torres (1997) Seminars in Immunol 9: 271-283; Donnelly et al. (1997) Annu Rev Immunol 15: 617-648; later herein]. Also, vaccination with RNA is an interesting and highly promising technology, cf. the above-mentioned reference by Deering R. P. et al.

Generally, plasmids can be fomulated in saline or acceptable buffer compositions such as Tyrode's buffer. Depending on the exact mode of administration, use of lipid delivery systems known in the art or fomulation in polymers such as poloxamines or poloxamers is preferred.

Useful formulations of nucleic acid vaccines are detailed in international patent application publication WO 2020/182901, and in the following patent applications: PCT/EP2020/087111, PCT/2021/059117, and EP21171313.3. In important aspects of the invention, DNA plasmids are formulated in Macrogolglycerol ricinoleate (also known as PEG-35 castor oil), a nonionic solubilizer and emulsifier made by reacting castor oil with ethylene oxide, and sold under the registered trademark Kolliphor®.

### 8^{th} Aspect of the Invention

The method of the 8^{th} aspect of the invention generally relates to induction of immunity and as such also entails methods that relate to treatment, prophylaxis and amelioration of disease.

When immunization methods entail that a fusion polypeptide of the invention or a composition comprising such a fusion polypeptide is administered the animal (e.g. the human) typically receives between 0.5 and 5,000 μg of the polypeptide of the invention per administration. The dosage can vary from protein to protein, but dosages between 5 and 1,000 μg are commonly used, and very often will the dosage be between 50 and 250 μg fusion protein.

In preferred embodiments of this aspect, the immunization scheme includes that the animal (e.g. the human) receives several immunizations, i.e. at least one repeated administration, such as a priming administration and one or more booster administrations. However, if a single immunization is found to provide for an effective protection, the immunization scheme can consist of such a single immunization; this is both highly patient compliant but also more economical.

Preferred embodiments of this aspect of the invention comprise that the administration is for the purpose of inducing protective immunity against the intracellular pathogen. In turn this means that the administration is a prophylactic or therapeutic treatment of whatever disease the intracellular pathogen is responsible for, i.e. the induced immune response reduces the risk of contracting disease caused by the intracellular pathogen, or the induced immune response reduces or eradicates disease cause by the intracellular pathogen.

Pharmaceutical compositions can as mentioned above comprise polypeptides, vectors, or nucleic acids of the invention. The pharmaceutical compositions will comprise a therapeutically effective amount thereof.

The term “therapeutically effective amount” or “prophylactically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. Reference is however made to the ranges for dosages of immunologically effective amounts of polypeptides, cf. above.

However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs/vectors in the individual to which it is administered. In some embodiments the invention comprises administering an immunogenically effective amount of a composition comprising at least one expression vector as defined and discussed herein with an effective dosage between 0.1 μg and 25 mg of the expression vector, such as between 0.5 μg and 20 mg, between 5 μg and 15 mg, between 50 μg and 10 mg, and between 500 μg and 8 mg, in particular about 0.0001, about 0.0005, about 0.001, about 0.005, about 0.01, about 0.05, about 0.1, about 0.5, about 1, about 2, about 3, about 4, about 5, about 6, about 7 and about 8 mg.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N. J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

As is apparent from the claims, the invention also relates to related aspect and embodiments to the treatment and prophylaxis disclosed herein: the invention also includes aspects and embodiments where
the fusion polypeptides of the invention is for use as a medicament, in particular for use in a method of the 8^{th} aspect of the invention; and
the nucleic acid fragment(s) of the invention or the vector of the invention is for use as a medicament, in particular for use in a method of the 8^{th} aspect of the invention.

### Example 1

Application of the APC DNA Targeting Technology with the Receptor Binding Domain from SARS-COV-2 as the B-Cell Rich Region

The aim of the studies were to compare the performance of the APC (CLL19) targeting DNA plasmid using the spike receptor binding domain (RBD) as the B-cell rich region delivered with Kolliphor® polymer by intra-muscular (IM) injection or by IM injection followed by electroporation (EP) with that of recombinant RBD (rRBD) adjuvanted with aluminium hydroxide (Al). The goal was to determine the ability to induce a humoral antigen specific antibody response, the ability of the generated antibodies that could interfere with infection cells by the virus in a micro-neutralization assay or a pseudo-neutralization assay, and the magnitude of the T lymphocyte response.

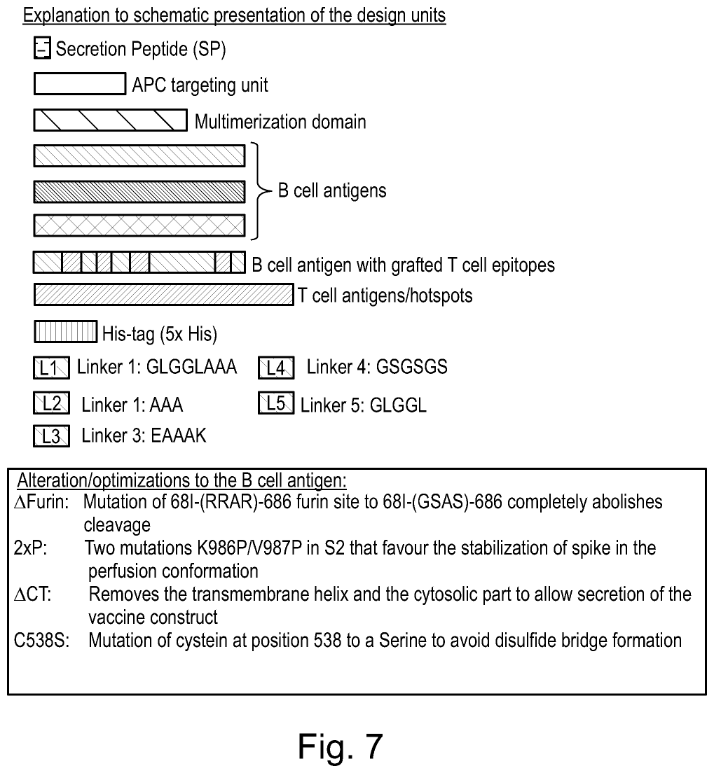

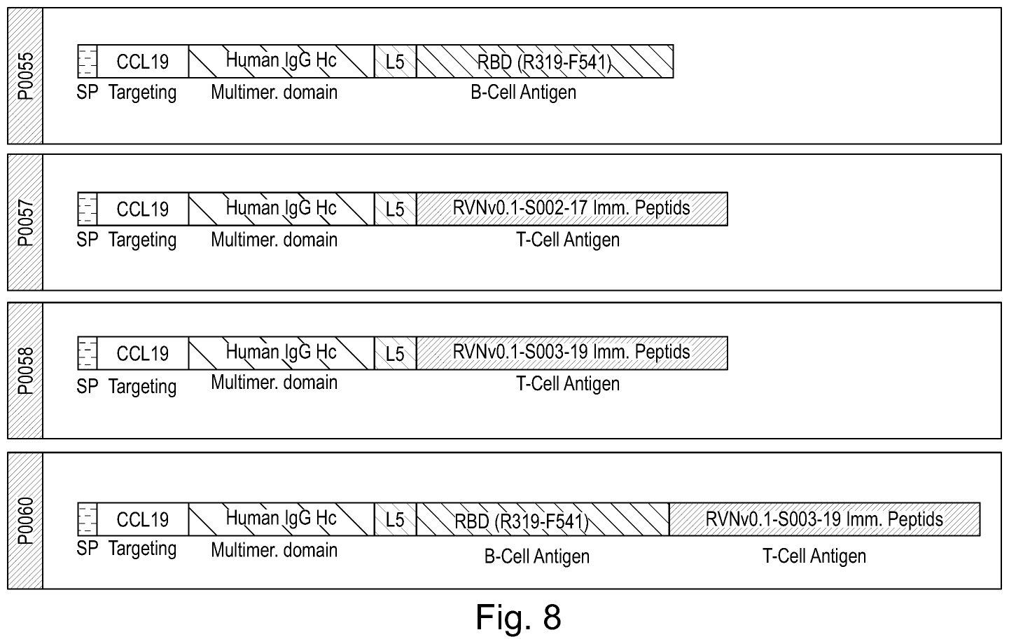

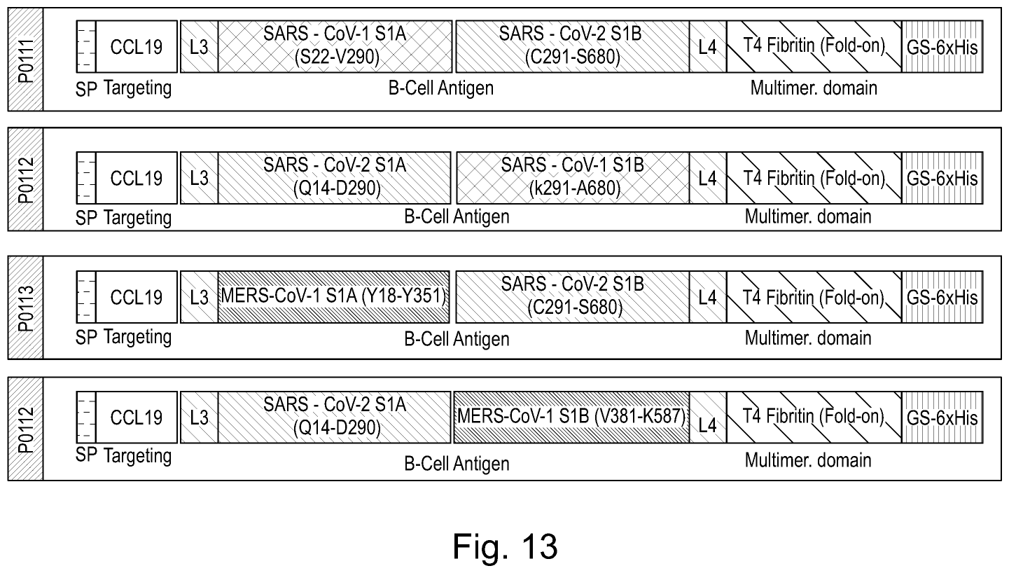

A number of vector designs were prepared, of which P0055 (see below) was evaluated in the current in vivo studies. The selected RBD was taken from full length SARS-CoV-2 Spike protein (Acc ID: YP-009724390.1) by excising amino acids Arg319 to Phe541. Plasmid vector designs of the invention are shown schematically in FIGS. 8-13, where FIG. 7 provide an explanation of the elements depicted in these figures.

Compared to the tested construct P0055, P0069 has been constructed with an aim to increase the expression level. Likewise, P0072 has been constructed with an aim to 1) increase expression levels, and 2) to form trimers instead of dimers of the expression product; this currently remains to be tested. P0080 is similar to P0055 with the exception of the addition of a His-tag. This has been tested in vivo to perform equally well as P0055 (data not shown).

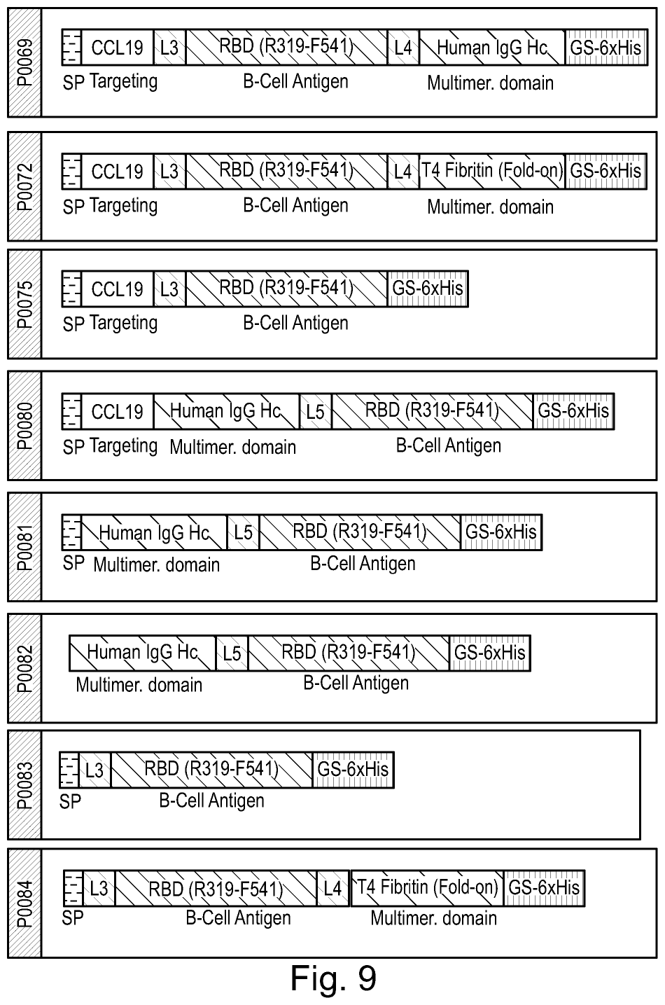

Several designs were created with the purpose as serving as controls. P0075 is designed as a control to investigate the effects of multimerization. P0081, P0083 and P0084 are controls for the effect of CCL19, and P0082 is a control for secretion (FIG. 9).

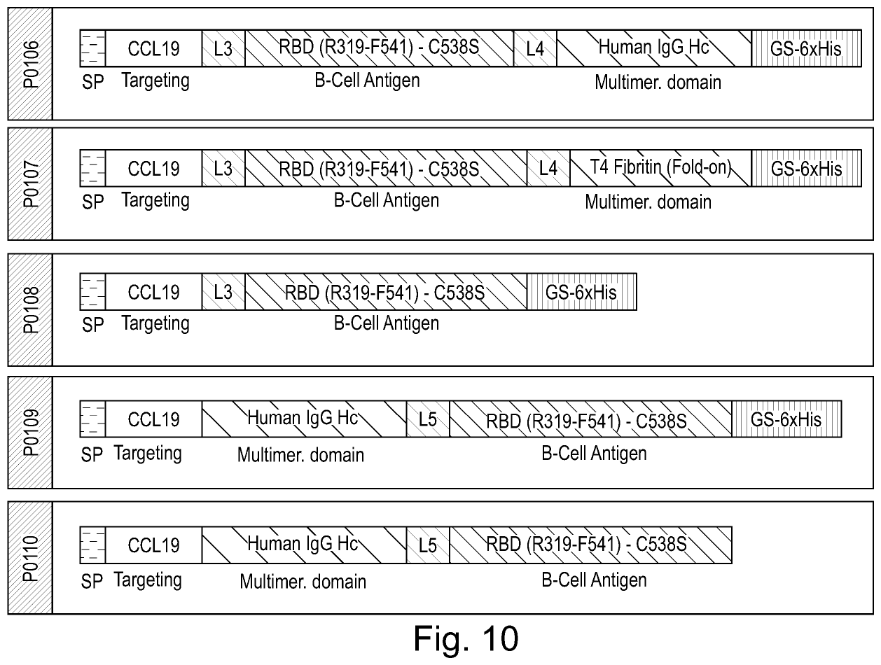

For further optimization of the RBD construct mutagenesis of potential problematic Cys538 to Ser on a number of designs P0055 (P0110), P0069 (P00106), P0072 (P00107), P0075 (P00108), P0080 (P00109) was done for better h1h4CH3 dimerization and T4 trimerization (FIG. 10).

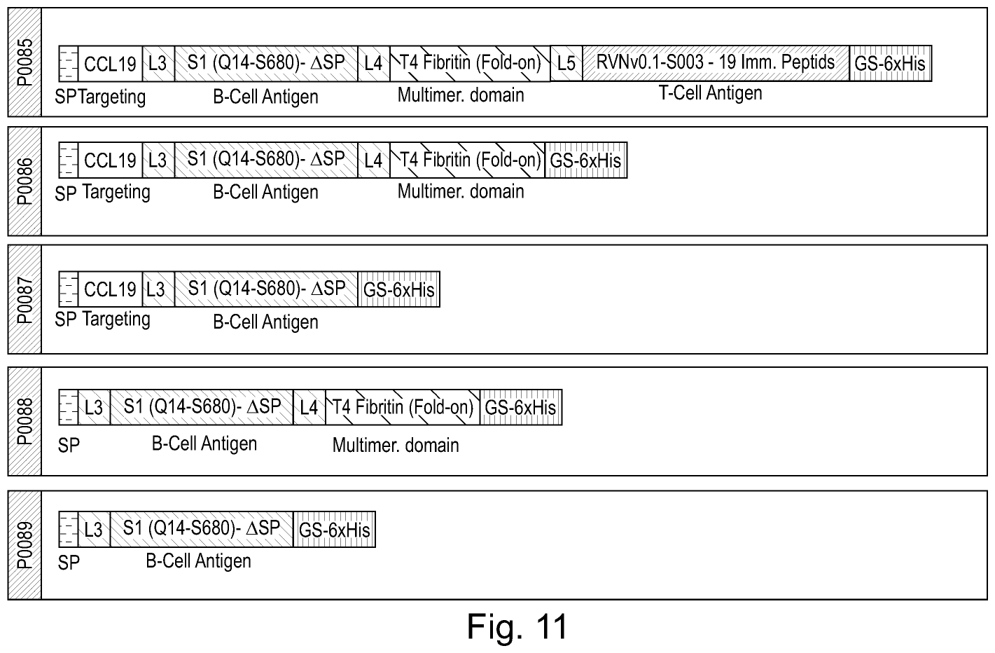

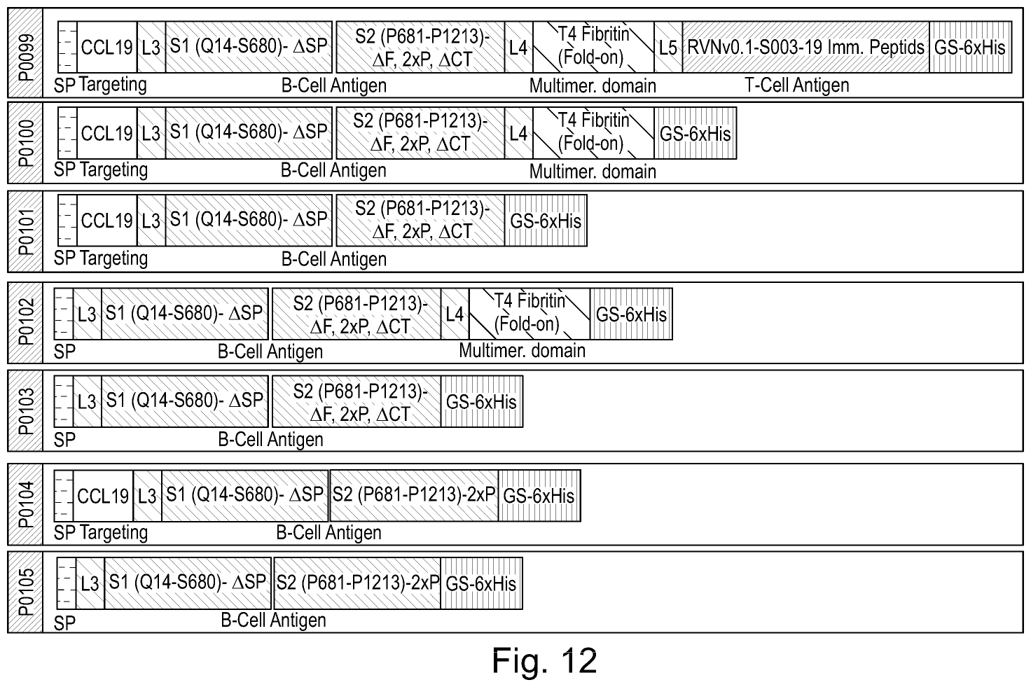

Several additional B cell designs have developed using either the S1 domain of the SARS-CoV-2 Spike protein (FIG. 11), the full-length spike protein (FIG. 12) or chimeric designs with N- and C-terminal domains (S1 and S2, respectively) from different coronavirus strains (ie. SARS-CoV-1, SARS-CoV-2, MERS-CoV-1).

All of the above described B cell rich designs containing CCL19 have been found to be secreted in vitro upon transfection of HEK293 cells using a CCL19 specific ELISA (data not shown).

The expression vector P0055 is shown schematically as a plasmid map in FIG. 4 and listed as SEQ ID NO: 23. The RBD sequence encoded by the plasmid is provided as SEQ ID NO 24, where it is linked to CC119 as an APC targeting unit via an IgG3 multimerization unit (the RBD is shown with underlining in SEQ ID NO: 24):

| P0055 (SEQ ID NO: 23): | |
|---|---|
| tggccattgc atacgttgta tccatatcat aatatgtaca tttatattgg ctcatgtcca | 60 |
| acattaccgc catgttgaca ttgattattg actagttatt aatagtaatc aattacgggg | 120 |
| tcattagttc atagcccata tatggagttc cgcgttacat aacttacggt aaatggcccg | 180 |
| cctggctgac cgcccaacga cccccgccca ttgacgtcaa taatgacgta tgttcccata | 240 |
| gtaacgccaa tagggacttt ccattgacgt caatgggtgg agtatttacg gtaaactgcc | 300 |
| cacttggcag tacatcaagt gtatcatatg ccaagtacgc cccctattga cgtcaatgac | 360 |
| ggtaaatggc ccgcctggca ttatgcccag tacatgacct tatgggactt tcctacttgg | 420 |
| cagtacatct acgtattagt catcgctatt accatggtga tgcggttttg gcagtacatc | 480 |
| aatgggcgtg gatagcggtt tgactcacgg ggatttccaa gtctccaccc cattgacgtc | 540 |
| aatgggagtt tgttttggca ccaaaatcaa cgggactttc caaaatgtcg taacaactcc | 600 |
| gccccattga cgcaaatggg cggtaggcgt gtacggtggg aggtctatat aagcagagct | 660 |
| cgtttagtga accgtcagat cgcctggaga cgccatccac gctgttttga cctccataga | 720 |
| agacaccggg accgatccag cctccgcggc cgggaacggt gcattggaac gcggattccc | 780 |
| cgtgccaaga gtgacgtaag taccgcctat agactctata ggcacacccc tttggctctt | 840 |
| atgcatgcta tactgttttt ggcttggggc ctatacaccc ccgcttcctt atgctatagg | 900 |
| tgatggtata gcttagccta taggtgtggg ttattgacca ttattgacca ctccaacggt | 960 |
| ggagggcagt gtagtctgag cagtactcgt tgctgccgcg cgcgccacca gacataatag | 1020 |
| ctgacagact aacagactgt tcctttccat gggtcttttc tgcagtcacc gtcgtcgacg | 1080 |
| gtatcgataa gcttgatatc gaattccgcc gccaccatgg ccccccgtgt gaccccactc | 1140 |
| ctggccttca gcctgctggt tctctggacc ttcccagccc caactctggg gggtgctaat | 1200 |
| gatgcggaag actgctgcct gtctgtgacc cagcgcccca tccctgggaa catcgtgaaa | 1260 |
| gccttccgct accttcttaa tgaagatggc tgcagggtgc ctgctgttgt gttcaccaca | 1320 |
| ctaaggggct atcagctctg tgcacctcct gaccagccct gggtggatcg catcatccga | 1380 |
| agactgaaga agtcttctgc caagaacaaa ggcaacagca ccagaaggag ccctgtgtct | 1440 |
| gagctcaaaa ccccacttgg tgacacaact cacacagagc ccaaatcttg tgacacacct | 1500 |
| cccccgtgcc caaggtgccc aggcggtgga agcagcggag gtggaagtgg aggacagccc | 1560 |
| cgagaaccac aggtgtacac cctgccccca tcccgggagg agatgaccaa gaaccaggtc | 1620 |
| agcctgacct gcctggtcaa aggcttctac cccagcgaca tcgccgtgga gtgggagagc | 1680 |
| agcgggcagc cggagaacaa ctacaacacc acgcctocca tgctggactc cgacggctcc | 1740 |
| ttcttcctct acagcaagct caccgtggac aagagcaggt ggcagcaggg gaacatcttc | 1800 |
| tcatgctccg tgatgcatga ggctctgcac aaccgcttca cgcagaagag cctctccctg | 1860 |
| tctccgggta aaggcctcgg tggcctggcg gccgcgaggg tgcagccgac tgagagtatc | 1920 |
| gttcgtttcc ctaacattac gaacctatgt ccgtttggag aggtatttaa tgctaccaga | 1980 |
| ttcgcatcag tctatgcttg gaacaggaaa cggatatcaa actgcgtggc cgactatagc | 2040 |
| gttctctaca acagtgcctc ttttagcact ttcaagtgct acggagtctc cccaaccaag | 2100 |
| ctaaacgacc tgtgcttcac aaacgtctac gctgactcct ttgtcatcag aggcgatgag | 2160 |
| gtgcggcaaa tcgcacctgg acaaactgga aaaatcgccg actataacta taagctgccc | 2220 |
| gatgacttta ctggatgcgt gatcgcttgg aattcaaaca atctcgacag taaggtagga | 2280 |
| ggcaattaca actaccttta tcggctcttc cgaaaatcaa acctgaaacc ctttgagcgt | 2340 |
| gatatctcga ctgaaattta ccaagctggc agcactcctt gcaacggagt ggagggcttc | 2400 |
| aattgttact ttcctctgca gagctacgga ttccaaccta caaacggggt aggctaccaa | 2460 |
| ccctaccgag tggtggtgct gagctttgag ctgctccatg ctccagcaac tgtatgcggc | 2520 |
| cctaagaagt caactaacct ggttaagaat aaatgcgtca acttttaggg atccagatct | 2580 |
| aacgacaaaa cgacaaaacg acaaggcgcc agatctggcg tttcgttttg tcgttttgtc | 2640 |
| gttagatctt tttccctctg ccaaaaatta tggggacatc atgaagcccc ttgagcatct | 2700 |
| gacttctggc taataaagga aatttatttt cattgcaata gtgtgttgga attttttgtg | 2760 |
| tctctcactc ggaaggacat atgggagggc aaatcattta aaacatcaga atgagtattt | 2820 |
| ggtttagagt ttggcaacat atgcccattc ttccgcttcc tcgctcactg actcgctgcg | 2880 |
| ctcggtcgtt cggctgcggc gagcggtatc agctcactca aaggcggtaa tacggttatc | 2940 |
| cacagaatca ggggataacg caggaaagaa catgtgagca aaaggccagc aaaaggccag | 3000 |
| gaaccgtaaa aaggccgcgt tgctggcgtt tttccatagg ctccgccccc ctgacgagca | 3060 |
| tcacaaaaat cgacgctcaa gtcagaggtg gcgaaacccg acaggactat aaagatacca | 3120 |
| ggcgtttccc cctggaagct ccctcgtgcg ctctcctgtt ccgaccctgc cgcttaccgg | 3180 |
| atacctgtcc gcctttctcc cttcgggaag cgtggcgctt tctcatagct cacgctgtag | 3240 |
| gtatctcagt tcggtgtagg tcgttcgctc caagctgggc tgtgtgcacg aaccccccgt | 3300 |
| tcagcccgac cgctgcgcct tatccggtaa ctatcgtctt gagtccaacc cggtaagaca | 3360 |
| cgacttatcg ccactggcag cagccactgg taacaggatt agcagagcga ggtatgtagg | 3420 |
| cggtgctaca gagttcttga agtggtggcc taactacggc tacactagaa gaacagtatt | 3480 |
| tggtatctgc gctctgctga agccagttac cttcggaaaa agagttggta gctcttgatc | 3540 |
| cggcaaacaa accaccgctg gtagcggtgg tttttttgtt tgcaagcagc agattacgcg | 3600 |
| cagaaaaaaa ggatctcaag aagatccttt gatcttttct acggggtctg acgctcagtg | 3660 |
| gaacgaaaac tcacgttaag ggattttggt catgagatta tcaaaaagga tcttcaccta | 3720 |
| gatcctttta aattaaaaat gaagttttaa atcaatctaa agtatatatg agtaaacttg | 3780 |
| gtctgacagt taccaatgct taatcagtga ggcacctatc tcagcgatct gtctatttcg | 3840 |
| ttcatccata gttgcctgac tcgggggggg ggggcgctga ggtctgcctc gtgaagaagg | 3900 |
| tgttgctgac tcataccagg cctgaatcgc cccatcatcc agccagaaag tgagggagcc | 3960 |
| acggttgatg agagctttgt tgtaggtgga ccagttggtg attttgaact tttgctttgc | 4020 |
| cacggaacgg tctgcgttgt cgggaagatg cgtgatctga tccttcaact cagcaaaagt | 4080 |
| tcgatttatt caacaaagcc gccgtcccgt caagtcagcg taatgctctg ccagtgttac | 4140 |
| aaccaattaa ccaattctga ttagaaaaac tcatcgagca tcaaatgaaa ctgcaattta | 4200 |
| ttcatatcag gattatcaat accatatttt tgaaaaagcc gtttctgtaa tgaaggagaa | 4260 |
| aactcaccga ggcagttcca taggatggca agatcctggt atcggtctgc gattccgact | 4320 |
| cgtccaacat caatacaacc tattaatttc ccctcgtcaa aaataaggtt atcaagtgag | 4380 |
| aaatcaccat gagtgacgac tgaatccggt gagaatggca aaagcttatg catttctttc | 4440 |
| cagacttgtt caacaggcca gccattacgc tcgtcatcaa aatcactcgc atcaaccaaa | 4500 |
| ccgttattca ttcgtgattg cgcctgagcg agacgaaata cgcgatcgct gttaaaagga | 4560 |
| caattacaaa caggaatcga atgcaaccgg cgcaggaaca ctgccagcgc atcaacaata | 4620 |
| ttttcacctg aatcaggata ttcttctaat acctggaatg ctgttttccc ggggatcgca | 4680 |
| gtggtgagta accatgcatc atcaggagta cggataaaat gcttgatggt cggaagaggc | 4740 |
| ataaattccg tcagccagtt tagtctgacc atctcatctg taacatcatt ggcaacgcta | 4800 |
| cctttgccat gtttcagaaa caactctggc gcatcgggct toccatacaa tcgatagatt | 4860 |
| gtcgcacctg attgcccgac attatcgcga gcccatttat acccatataa atcagcatcc | 4920 |
| atgttggaat ttaatcgcgg cctcgagcaa gacgtttccc gttgaatatg gctcataaca | 4980 |
| ccccttgtat tactgtttat gtaagcagac agttttattg ttcatgatga tatattttta | 5040 |
| tottgtgcaa tgtaacatca gagattttga gacacaacht ggetttcccc ccccccccat | 5100 |
| tattgaagca tttatcaggg ttattgtctc atgagcggat acatatttga atgtatttag | 5160 |
| aaaaataaac aaataggggt tccgcgcaca tttccccgaa aagtgccacc tgacgtctaa | 5220 |
| gaaaccatta ttatcatgac attaacctat aaaaataggc gtatcacgag gccctttcgt | 5280 |
| ctcgcgcgtt tcggtgatga cggtgaaaac ctctgacaca tgcagctccc ggagacggtc | 5340 |
| acagcttgtc tgtaagcgga tgccgggagc agacaagccc gtcagggcgc gtcagcgggt | 5400 |
| gttggcgggt gtcggggctg gcttaactat gcggcatcag agcagattgt actgagagtg | 5460 |
| caccatatgc ggtgtgaaat accgcacaga tgcgtaagga gaaaataccg catcagattg | 5520 |
| gctat | 5525 |
| APC-IqG3-SARS-CoV-2 RBD (SEQ ID NO: 24): | |
| MAPRVTPLLA FSLLVLWTFP APTLGGANDA EDCCLSVTQR PIPGNIVKAF RYLLNEDGCR | 60 |
| VPAVVFTTLR GYQLCAPPDQ PWVDRIIRRL KKSSAKNKGN STRRSPVSEL KTPLGDTTHT | 120 |
| EPKSCDTPPP CPRCPGGGSS GGGSGGQPRE PQVYTLPPSR EEMTKNQVSL TCLVKGFYPS | 180 |
| DIAVEWESSG QPENNYNTTP PMLDSDGSFF LYSKLTVDKS RWQQGNIFSC SVMHEALHNR | 240 |
| FTQKSLSLSP GKGLGGLAAA RVQPTESIVR FPNITNLCPF GEVFNATRFA SVYAWNRKRI | 300 |
| SNCVADYSVL YNSASFSTEK CYGVSPTKLN DLCFTNVYAD SFVIRGDEVR QIAPGQTGKI | 360 |
| ADYNYKLPDD FTGCVIAWNS NNLDSKVGGN YNYLYRLFRK SNLKPFERDI STEIYQAGST | 420 |
| PCNGVEGFNC YFPLQSYGFQ PTNGVGYQPY RVVVLSFELL HAPATVCGPK KSTNLVKNKC | 480 |
| VNF | 483 |

### Animal Study Details

Three different mice studies in two different mouse strains were conducted to evaluate P0055.

In study A, BALB/c mice immunized IM with one, two, three or five doses of 25 ug P0055 plasmid formulated with 3% w/v Kolliphor® polymer. Mice immunized subcutaneously (SC) twice with 10 μg rRBD+2% Alhydrogel served as positive controls. Mock immunized (i.e. DNA plasmid without spike protein; P0053_PGTV4_CCL19; five doses of 25 ug) and placebo (2% Alhydrogel) served as negative controls.

8 groups (termed 1-8) of mice were assigned to the following treatments:
1. Plasmid P0055 (25 μg) (0 wk), n=10
2. Plasmid P0055 (25 μg) (0,4 wk), n=10
3. Plasmid P0055 (25 μg) (0, 2, 4 wk), n=10
4. Plasmid P0055 (25 μg) (0, 1, 2, 3, 4 wk), n=10
5. Plasmid P0055 (25 μg)+P0058 (25 μg (0, 2, 4 wk), n=10
6. RBD Protein+Alhydrogel 2%, (10 μg), n=9
7. Alhydrogel 2% placebo, n=5
8. Plasmid P0053—pTGV-CCL19—Mock, (25 μg) (0, 1, 2, 3, 4 wk), n=5

Mice were terminated 28 days after last immunization. Read-outs were body weight variation (based on 3 weekly measurements) and measurements based on blood sampling (day 13, day 27, day 42 and day 56) and spleen sampling (at day 56). Measurement performed were determination of anti-RBD IgG in blood, splenic T-cell activation (IFN-γ release ELISPOT on spleen cells restimulated with peptides), and neutralization of live SARS-CoV-2 in vitro (SARS-CoV-2 2019 nCOV ITALY/INMI1; MNt-CPE assay).

In study B, C57BL/6 mice were immunized IM with 25 μg, 50 μg, 100 μg with P0055 μlasmid formulated with 3% w/v Kolliphor® polymer as one or two doses (day 0, and day 0,28). Mice immunized five time times (1-week intervals) with 25 μg P0055 μlasmid or rRBD+2% Alhydrogel (two-dose; SC) served as positive controls. Mock immunized (P0053_PGTV4_CCL19; Five doses of 100 ug) and placebo (2% Alhydrogel; two doses; SC) served as negative controls (data not shown).

10 groups (termed 1-10) of mice were assigned to the following treatments:
1. rRBD Protein+Alhydrogel 2%, (10 μg; 0,28), n=10 s.c.
2. P0055 (25 μg; 28), n=10
3. P0055 (25 μg; 0,28), n=10
4. P0055 (25 μg; 0,7,14,21,28), n=7
5. P0055 (50 μg; 28), n=7
6. P0055 (50 μg; 0,28), n=7
7. P0055 (100 μg; 28), n=7
8. P0055 (100 μg; 0,28), n=7
9. P0053 (100 μg; 0,28), n=7
10. Alhydrogel 2% placebo (0,28). n=7

Mice were terminated 28 days after last immunization. Sera and spleens were collected and analysed for total anti-RBD-IgG, neutralizing effect of antibodies against live virus (SARS-CoV-2 2019 nCOV ITALY/INMI1; MNt-CPE assay). Spleenocytes were analyzed for their capacity to elicit an IFN-γ response in ELISPOT. The T-cells were re-stimulated with either peptides or concanavalin A (conA, positive control for IFN-γ release; data not shown) or irrelevant peptide (negative control; data not shown). Pools 1, 2, and 3 are constituted by overlapping peptides from the RBD protein according to the following distribution:
Pool RBD1: CoV2_s-319, CoV2_s-325, CoV2_s-331, CoV2_s-337, CoV2_s-343, CoV2_s-349, CoV2_s-355, CoV2_s-361, CoV2_s-367, CoV2_s-373, CoV2_s-379, CoV2_s-385
Pool RBD2: CoV2_s-391, CoV2_s-397, CoV2_s-403, CoV2_s-409, CoV2_s-415, CoV2_s-421, CoV2_s-427, CoV2_s-439, CoV2_s-445, CoV2_s-451, CoV2_s-457
Pool RBD3: CoV2_s-463, CoV2_s-469, CoV2_s-475, CoV2_s-481, CoV2_s-487, CoV2_s-493, CoV2_s-499, CoV2_s-505, CoV2_s-511, CoV2_s-517, CoV2_s-523, CoV2_s-529

In study C, C57BL/6 mice immunized with two doses of 100 μg with P0055_CCL19_RBD_PoC plasmid or P0167_CCL3_RBD (0,28) formulated in 1×PBS by intra-muscular injection followed by electroporation (EP), or IM injection of plasmid formulated with 3% w/v Kolliphor® polymer. Mock immunized (P0053_PGTV4_CCL19; two doses of 100 μg; EP) served as negative control. Mice were terminated 14 days after last immunization. Sera were collected and analysed for total anti-RBD-IgG, neutralizing effect of antibodies against by pseudo-neutralization assay (MSD). In the P0167_CCL3_RBD construct the native secretion signal was exchanged with the CCL19 secretion peptide to obtain equal expression of the two constructs for a more direct comparison of the APC targeting units.

### Results

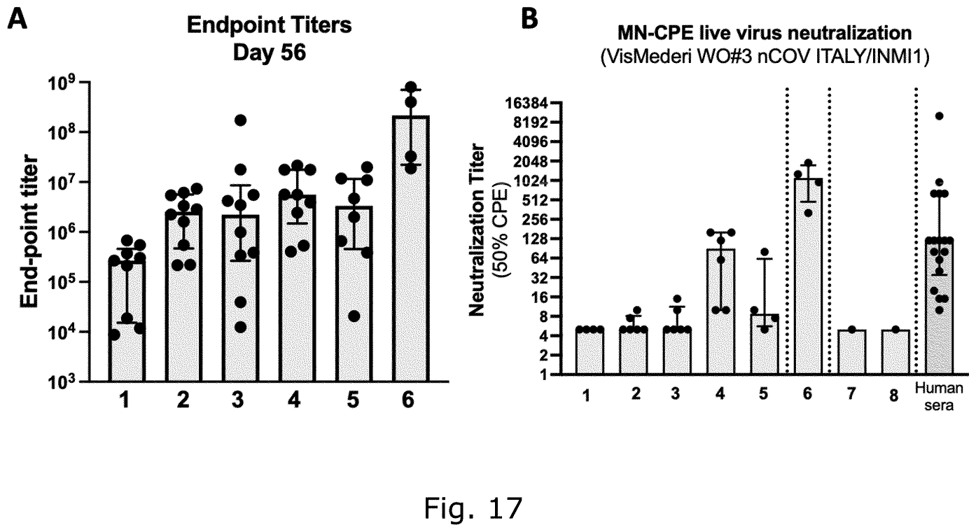

For study A, FIG. 17A show a bar graph of the ELISA measurements of total RBD specific IgG levels shown as End-point titer levels for individual mice. Immunization with P0055 CCL-19 RBD shows successful extracellular expression of the RBD protein in the immunized mice accompanied by a resulting potent humoral response with antibody titers. No impact on the humoral response was shown by co-administration of the T cell epitope plasmid design P0058 (Gr. 5). FIG. 17B shows a bar graph visualizing the neutralizing effect of antibodies in the tested groups and antibodies from convalescent patient sera. As is clear, the mice from group 4 produces antibodies that are comparable in neutralizing efficacy with those produced by convalescent patients. This supports the application of APC targeting with the spike protein or fragments thereof within in the field of corona virus vaccine technology. Increasing the number of doses had a positive impact on both IgG titers and neutralizing capacity.

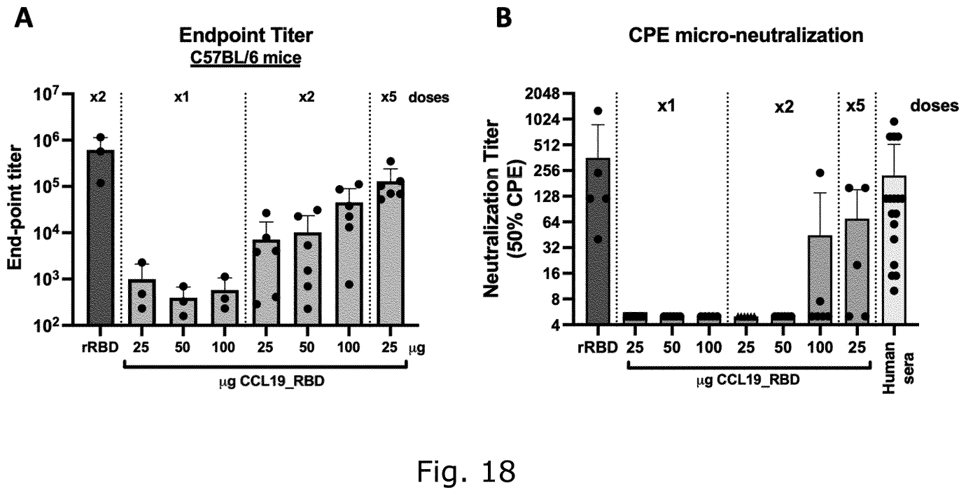

For study B, FIG. 18A show the ELISA measurements of total RBD specific IgG levels for individual mice. Like for the BALB/c mouse in study A, a potent humoral response was shown in C57BL/6 mice, showing in vivo successful extracellular expression of the RBD protein from the P0055 μlasmid. FIG. 18B show the neutralizing effect of antibodies in the tested groups and antibodies from convalescent patient sera. Increasing the concentration of administered P0055 had a positive effect on the humoral response, showing that administration of 100 μg as two doses was comparable to delivery of 25 μg in a five-dose regime.

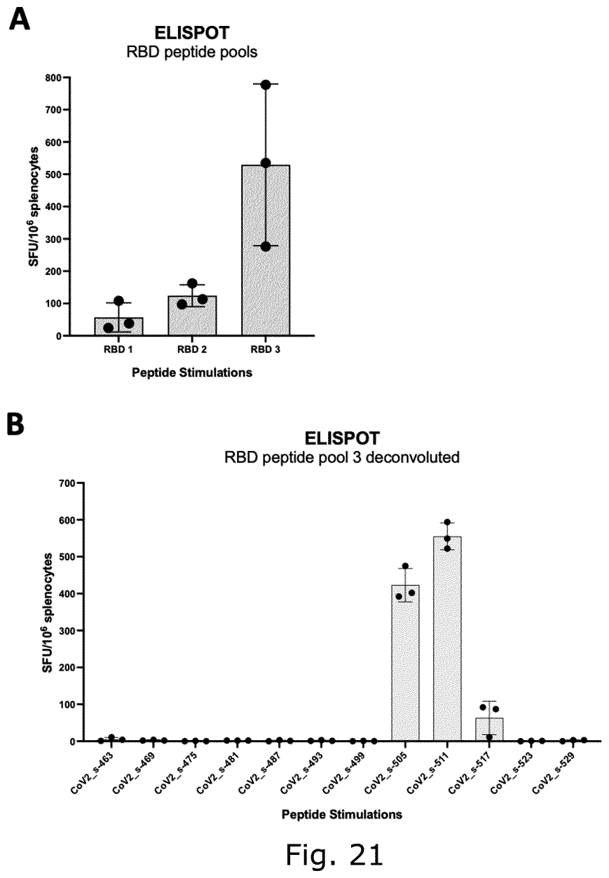

In addition, FIG. 21 show bar graphs visualizing the IFN-γ release from the T-cells of mice from group 4. Cells stimulated with RBD pool 2 and 3 were found to exhibit a higher release of IFN-γ. Deconvolution of RBD peptide pool 3 show that the IFN-γ response is mainly driven by a single hotspot.

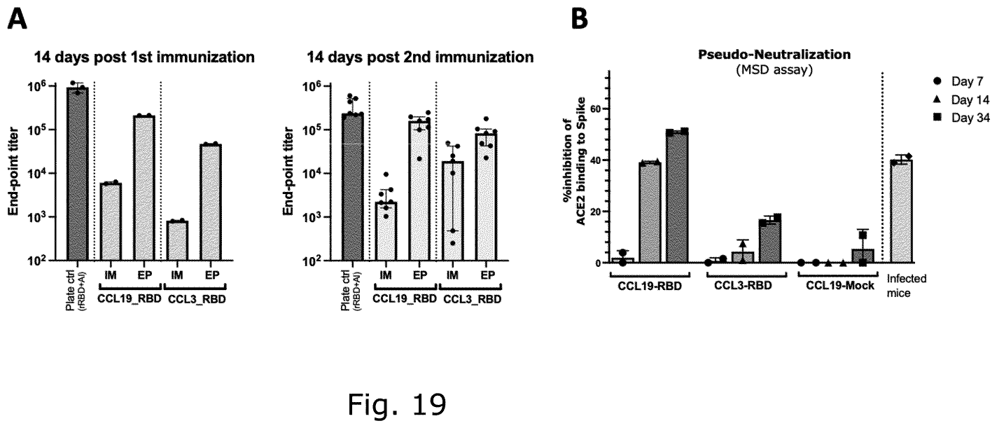

For study C, FIG. 19A show two bar graphs of the ELISA measurements of total RBD specific IgG levels in sera isolated two weeks after first immunization (left bar graph; pooled sera) and two weeks after the booster immunization (right bar graph; individual mice) compared to a plate control consisting of sera from mice immunized with 10 μg rRBD+AI from a separate study. FIG. 19B show the neutralizing effect of antibodies in pooled sera isolated at day 7, day 14 and day 34 from the immunized mice and sera from convalescent mice sera (i.e. K18-hACE2 C57BL/6J mice surviving challenge with TCID50 dose corresponding to LD50 of SARS-CoV-2 (WA1/USA 2020)) measured in a pseudo-neutralizing assay (MSD). Overall, immunization by electroporation (EP) resulted in high level antibodies with high neutralizing capacity against RBD already after one immunization. Both DNA constructs with CCL19 and CCL3 as APC targeting units were able to induce anti-RBD antibodies, although the level of neutralizing antibodies was significantly higher when utilizing CCL19.

### CONCLUSIONS

A humoral response can be induced when using the Spike RBD design as a B-cell rich region, according to the present invention in same magnitude as with recombinant Spike RBD+2% Alum. The induced antibody titers were furthermore highly functional, showing a neutralization efficiency comparable to convalescent patient and mouse sera. The constructs of the present invention in addition showed a strong T cell response using a spike RBD design, although the response may be limited to a few epitopes.

### Example 2

Application of the DNA Targeting Technology with a T-Cell Rich Region Designed and Predicted with Machine Learning Technology

The aim of the study was to investigate the immunogenicity of a T-cell rich region designed using machine learning technology, delivered to animals using the APC (CCL19) targeting DNA plasmid and the Kolliphor® polymer. The goal was to determine the magnitude and breadth of the T cell response to identify correctly predicted highly immunogenic hotspots with a suitable density of T-cell epitopes for a productive response.

A variety of different strategies were employed to design T-cell epitope-rich DNA-inserts, of which P0057 was evaluated in the current in vivo study.

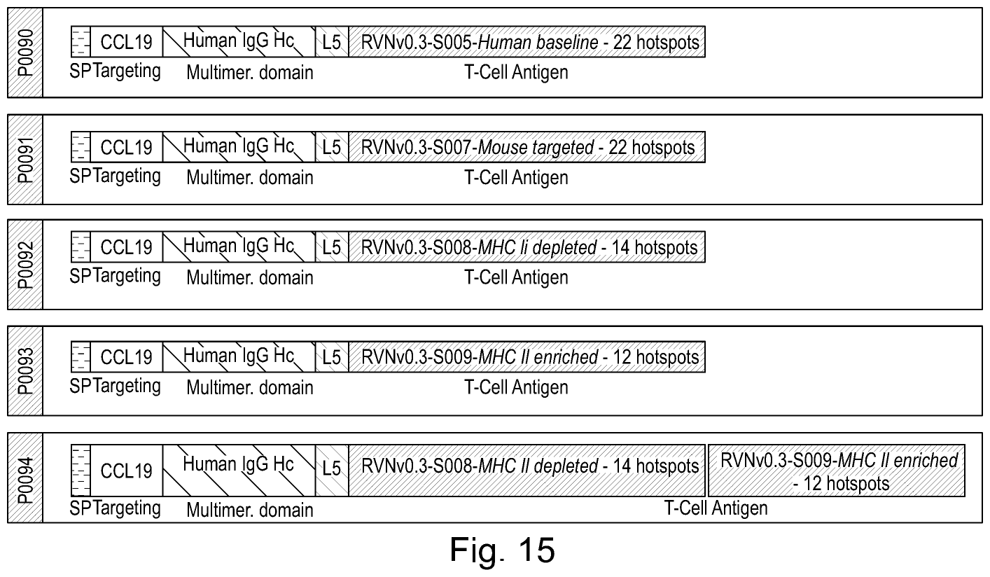

Other strategies include designs that evaluate the capability of the machine learning technology to elicit broad protection by including mice as a sub-population of the human population (P0090) compared to a mouse specific strategy (P0091), and designs to study the effect of having different balance of MHC class I and MHC class II epitopes in the T cell rich regions (P0092, P0093 and P0094). A schematic overview of the designs can be found in FIG. 15. These constructs have been shown to be expressed and secreted in vitro after HEK293 cell transfection (data not shown).

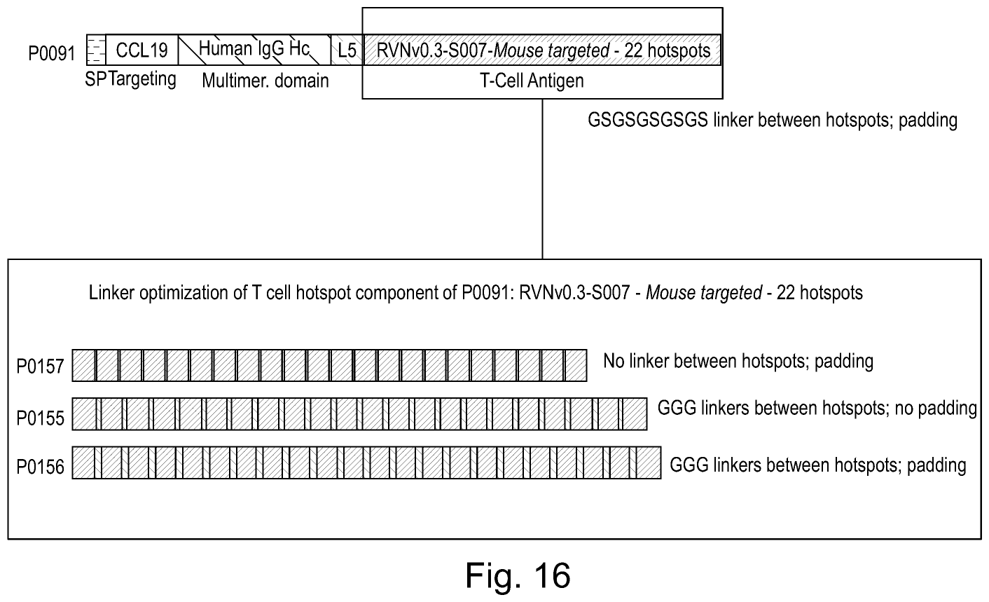

Changing the length of the linkers between the inserted T cell hotspots can allow for more real state and thus insertion of additional epitopes. Designs to explore this have been constructed and are shown as schematic representations in FIG. 16.

Another strategy relied on epitope grafting, which is a process of engineering the sequence of the spike protein by replacing stretches of amino acids with peptides that are predicted to be T-cell epitopes. The different grafting approaches employ different means of identifying potential graft candidates from a pool of predicted epitopes (termed graft candidates) by comparing the sequence of the epitopes to the wildtype sequence of the spike protein. The first grafting approach relied on sequence identity, only performing grafts if the graft candidate could be aligned to the wildtype sequence with a maximum of two mismatches in the amino acid sequence (multiple sequence alignment; MSA). The second grafting approach used a deep neural network latent variable model to perform grafts. We employed a deep Markov model (BIFROST) trained to generate local protein structure (Thygesen, Christian B., et al. “Efficient Generative Modelling of Protein Structure Fragments using a Deep Markov Model.” International Conference on Machine Learning. PMLR, 2021) by encoding the sequences of graft candidates as well as the wildtype into distributions over latent variables.

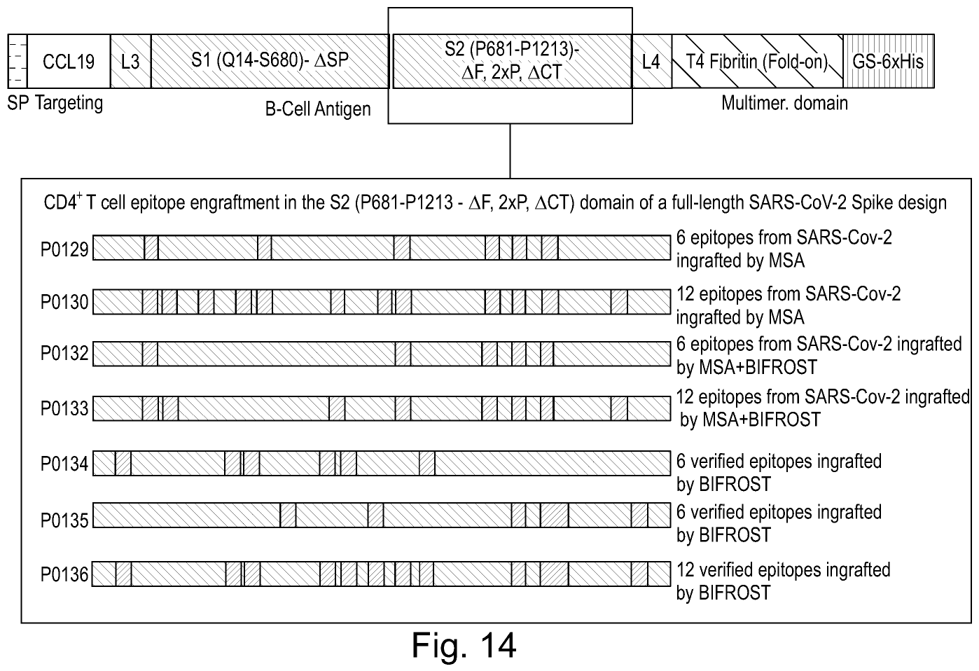

Grafting was performed if KL-divergence between the obtained distributions were less than 13. This second approach is called BIFROST. The third grafting approach was a combination of the first two, wherein grafts were performed using the MSA approach, and then evaluated by BIFROST (MSA+BIFROST). If BIFROST identified grafts with KL-divergence over 13, the sequence was reverted to the wildtype. Grafts were restricted to the S2-domain of the SARS-CoV-2 full length spike protein, which encodes the stem domain. Additionally, grafts were not allowed in the mutated furin cleavage site nor at the 2P mutation site. A schematic presentation of the grafted construct can be visualized in FIG. 14.

The grafted constructs have not yet been evaluated in vivo, but have been found to be expressed and secreted to the surround supernatant in vitro, when introduced into HEK293 cells by transfection. The table below show expression level measured by CCL19 ELISA of cell culture supernatant. As expected, constructs without CCL19 was not detected (indicated by

| | | CCL19 |
|---|---|---|
| Plasmid | | ELISA |
| ID | Plasmid description** | (pg/mL) |
| P0004* | pTVG4 empty plasmid backbone | 26.30 |
| P0080 | pTVG4_mccl19_IgG3_RBD | 843.30 |
| P0099 | mccl19_FL_SpikedF2P_T4_RVNS03 | 350.23 |
| P0100 | mccl19_FL_SpikedF2P_T4 | 370.66 |
| P0102* | mSP_FL_SpikedF2P_T4 | 24.45 |
| P0103* | mSP_FL_SpikedF2P | 24.74 |
| P0104 | mccl19_FL_Spike2P | 301.33 |
| P0105* | mSP_FL_Spike2P | 25.60 |
| P0117* | mSP_FL_SpikedF2P_T4_GSOpt | 26.03 |
| P0118 | mccl19_FL_SpikedF2P_T4_GSOpt | 119.38 |
| P0129 | mccl19_FL_SpikedF2P- | 56.10 |
| | SARS-CoV-2_TOP6_T4_GSOpt | |
| P0130 | mccl19_FL_SpikedF2P- | 54.23 |
| | SARS-CoV-2_TOP12_T4_GSOpt | |
| P0132 | mccl19_FL_SpikedF2P- | 78.10 |
| | SARS-CoV-2_TOP6-bifrost- | |
| | salvaged_T4_GSOpt | |
| P0133 | mccl19_FL_SpikedF2P- | 82.42 |
| | SARS-CoV-2_TOP12-bifrost- | |
| | salvaged_T4_GSOpt | |
| P0134 | mccl19_FL_SpikedF2P-bifrost- | 95.94 |
| | verified-6_T4_GSOpt | |
| P0135 | mccl19_FL_SpikedF2P-bifrost- | 64.58 |
| | verified-6-duplicate_T4_GSOpt | |
| P0136 | mccl19_FL_SpikedF2P-bifrost- | 58.97 |
| | verified-12_T4_GSOpt | |

*The construct does not contain CCL19 and is thus not detected in the supernatant.
**Abbreviations: mccl19: mouse CCL19; mSP: mouse secretion peptide; FL: Full length; SpikedF2P: Spike modified with ΔFurin and insertion of two proline residues; T4: T4 fibritin fold-on domain for trimerization; RBD: receptor binding domain; RVNS03: RAVEN strategy 03 for T cell epitope prediction; GSOpt: Codon optimization of the construct; TOP6/12: insertion of the top 6/12 T cell epitopes by either MSA: multiple sequence alignment or BIFROST.

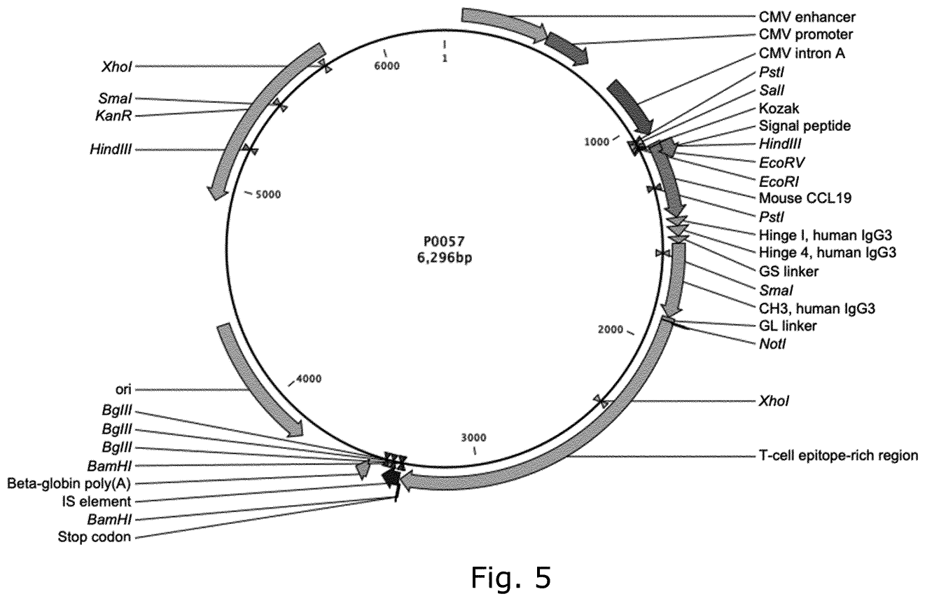

The proof-of-concept plasmid (1P0057, cf. FIG. 5 and SEQ ID NO: 42) was tested in vivo in mice studies. T cell epitopes of the MHCI and MHCII type predicted to bind to C57BL/6 mouse MHC were identified across the SARS-CoV-2 genome, according to algorithms similar to those previously published (Jurtz V et al. The Journal of Immunology (2017) ji1700893; DOT: 10.4049/jimmunol.1700893; Reynisson et al. Nucleic Acids Research, May 2020, https://doi.org/10.1093/nar/gkaa379). An evolutionary algorithm was then applied with a fitness function aimed to identify the set of T-cell hotspots (length: 35 AA), that in combination would have the highest T-cell epitope density when utilising ˜400 amino acid real estate. The selected hotspots are listed in the following table:

| | | | SARS- | | | |
|---|---|---|---|---|---|---|
| | | | CoV-2 | | | |
| n | np_id | neo_seq | gene | pos | n_mhc1 | n_mhc2 |
| 1 | 1ab_1532 | (SEQ ID NO: 25) | 1ab | 1532 | 15 | 2 |
| | | KSVYYTSNPTTFHLDGEVITFDNLKTLL | | | | |
| 2 | 1ab_2510 | (SEQ ID NO: 26) | 1ab | 2510 | 9 | 0 |
| | | KTYERHSLSHFVNLDNL | | | | |
| 3 | 1ab_2551 | (SEQ ID NO: 27) | 1ab | 2551 | 12 | 0 |
| | | SSAKSASVYYSQLMCQPILLL | | | | |
| 4 | 1ab_3941 | (SEQ ID NO: 28) | 1ab | 3941 | 11 | 13 |
| | | QAIASEFSSLPSYAAFATAQEAYEQAVA | | | | |
| 5 | 1ab_5157 | (SEQ ID NO: 29) | 1ab | 5157 | 15 | 3 |
| | | FNSTYASQGLVASIKNFKSVLYYQNNVFM | | | | |
| 6 | 1ab_5576 | (SEQ ID NO: 30) | 1ab | 5576 | 10 | 6 |
| | | YPTLNISDEFSSNVANYQKVGMQKYSTL | | | | |
| 7 | 1ab_6605 | (SEQ ID NO: 31) | 1ab | 6605 | 9 | 0 |
| | | SVGPKQASLNGVTLI | | | | |
| 8 | 1ab_6839 | (SEQ ID NO: 32) | 1ab | 6839 | 12 | 0 |
| | | MNVAKYTQLCQYLNTLTL | | | | |
| 9 | 3a_31 | (SEQ ID NO: 33) | 3a | 31 | 6 | 0 |
| | | TATIPIQASLPFGWLI | | | | |
| 10 | 3a_86 | (SEQ ID NO: 34) | 3a | 86 | 9 | 0 |
| | | FVTVYSHLLLVAAGLEAPFLYL | | | | |
| 11 | 6_21 | (SEQ ID NO: 35) | 6 | 21 | 3 | 0 |
| | | FKVSIWNLDYI | | | | |
| 12 | 7a_102* | (SEQ ID NO: 36) | 7a | 102 | 4 | 0 |
| | | IVAAIVFITLC | | | | |
| 13 | 8_12 | (SEQ ID NO: 37) | 8 | 12 | 3 | 0 |
| | | VAAFHQECSL | | | | |
| 14 | M_36 | (SEQ ID NO: 38) | M | 36 | 9 | 0 |
| | | FAYANRNRFLYIIKL | | | | |
| 15 | N_297 | (SEQ ID NO: 39) | N | 297 | 8 | 7 |
| | | YKHWPQIAQFAPSASAFFGMSRI | | | | |
| 16 | S_324 | (SEQ ID NO: 40) | S | 324 | 7 | 0 |
| | | SIVRFPNITNL | | | | |
| 17 | S_501 | (SEQ ID NO: 41) | S | 501 | 9 | 0 |
| | | GVGYQPYRVVVLSFELL | | | | |

* The peptide 7a_102 could not be purified and was not used for evaluation.

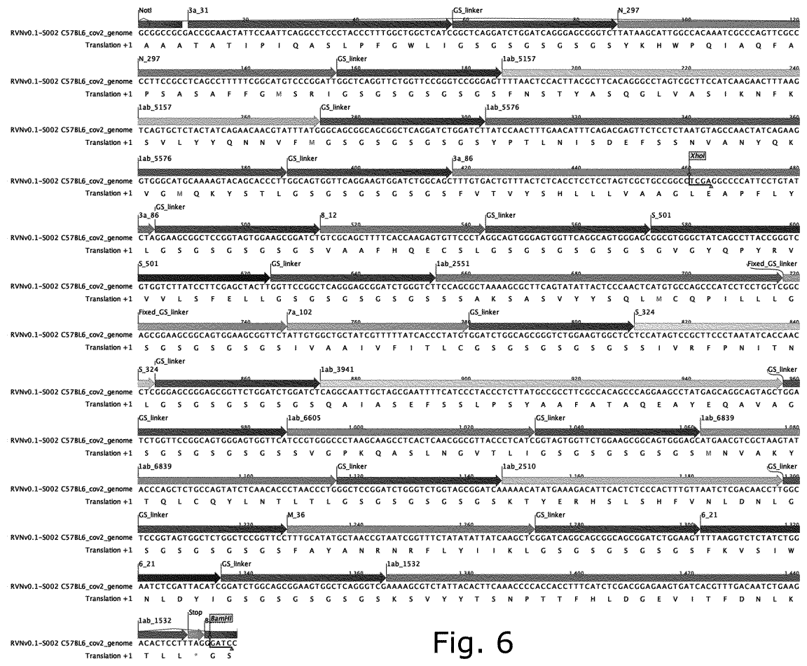

The selected hot spots where then linked together using high flexible 5×GS linkers in a final T-cell rich design as illustrated in FIG. 6. A schematic presentation of the design is found in FIG. 8.

The final vector design was made utilizing the CCL19 chemokine as an APC targeting domain, and the human IgG3 Heavy chain fragment as a dimerization. The final expression vector P0057 is shown as a plasmid map in FIG. 5 and listed as SEQ ID NO: 42. The fusion polypeptide sequence containing the T-cell epitope-rich region (FIG. 6, up to the C-terminal Leu) encoded by the plasmid is provided as SEQ ID NO: 43, residues 258-740 (underlined in SEQ ID NO: 43). The entire nucleotide sequence in FIG. 6 is provided in SEQ ID NO: 42 (see below, where the coding region, i.e. residues 1888-1450, for the T-cell epitope-rich region is underlined.

| P0057 (SEQ ID NO: 42): | |
|---|---|
| tggccattgc atacgttgta tccatatcat aatatgtaca tttatattgg ctcatgtcca | 60 |
| acattaccgc catgttgaca ttgattattg actagttatt aatagtaatc aattacgggg | 120 |
| tcattagttc atagcccata tatggagttc cgcgttacat aacttacggt aaatggcccg | 180 |
| cctggctgac cgcccaacga cccccgccca ttgacgtcaa taatgacgta tgttcccata | 240 |
| gtaacgccaa tagggacttt ccattgacgt caatgggtgg agtatttacg gtaaactgcc | 300 |
| cacttggcag tacatcaagt gtatcatatg ccaagtacgc cccctattga cgtcaatgac | 360 |
| ggtaaatggc ccgcctggca ttatgcccag tacatgacct tatgggactt tcctacttgg | 420 |
| cagtacatct acgtattagt catcgctatt accatggtga tgcggttttg gcagtacatc | 480 |
| aatgggcgtg gatagcggtt tgactcacgg ggatttccaa gtctccaccc cattgacgtc | 540 |
| aatgggagtt tgttttggca ccaaaatcaa cgggactttc caaaatgtcg taacaactcc | 600 |
| gccccattga cgcaaatggg cggtaggcgt gtacggtggg aggtctatat aagcagagct | 660 |
| cgtttagtga accgtcagat cgcctggaga cgccatccac gctgttttga cctccataga | 720 |
| agacaccggg accgatccag cctccgcggc cgggaacggt gcattggaac gcggattccc | 780 |
| cgtgccaaga gtgacgtaag taccgcctat agactctata ggcacacccc tttggctctt | 840 |
| atgcatgcta tactgttttt ggcttggggc ctatacaccc ccgcttcctt atgctatagg | 900 |
| tgatggtata gcttagccta taggtgtggg ttattgacca ttattgacca ctccaacggt | 960 |
| ggagggcagt gtagtctgag cagtactcgt tgctgccgcg cgcgccacca gacataatag | 1020 |
| ctgacagact aacagactgt tcctttccat gggtcttttc tgcagtcacc gtcgtcgacg | 1080 |
| gtatcgataa gcttgatatc gaattccgcc gccaccatgg ccccccgtgt gaccccactc | 1140 |
| ctggccttca gcctgctggt tctctggacc ttcccagccc caactctggg gggtgctaat | 1200 |
| gatgcggaag actgctgcct gtctgtgacc cagcgcccca tccctgggaa catcgtgaaa | 1260 |
| gccttccgct accttcttaa tgaagatggc tgcagggtgc ctgctgttgt gttcaccaca | 1320 |
| ctaaggggct atcagctctg tgcacctcct gaccagccct gggtggatcg catcatccga | 1380 |
| agactgaaga agtcttctgc caagaacaaa ggcaacagca ccagaaggag ccctgtgtct | 1440 |
| gagctcaaaa ccccacttgg tgacacaact cacacagagc ccaaatcttg tgacacacct | 1500 |
| cccccgtgcc caaggtgccc aggcggtgga agcagcggag gtggaagtgg aggacagccc | 1560 |
| cgagaaccac aggtgtacac cctgccccca tcccgggagg agatgaccaa gaaccaggtc | 1620 |
| agcctgacct gcctggtcaa aggcttctac cccagcgaca tcgccgtgga gtgggagagc | 1680 |
| agcgggcagc cggagaacaa ctacaacacc acgcctccca tgctggactc cgacggctcc | 1740 |
| ttcttcctct acagcaagct caccgtggac aagagcaggt ggcagcaggg gaacatcttc | 1800 |
| tcatgctccg tgatgcatga ggctctgcac aaccgcttca cgcagaagag cctctccctg | 1860 |
| tctccgggta aaggcctcgg tggcctggcg gccgcgaccg caactattcc aattcaggcc | 1920 |
| tccctaccct ttggctggct catcggctca ggatctggat cagggagcgg gtcttataag | 1980 |
| cattggccac aaatcgccca gttcgcccct tccgcctcag cctttttcgg catgtcccgg | 2040 |
| attggctcag gttctggttc cgggtccggg agttttaact ccacttacgc ttcacagggc | 2100 |
| ctagtcgctt ccatcaagaa ctttaagtca gtgctctact atcagaacaa cgtatttatg | 2160 |
| ggcagcggca gcggctcagg atctggatct tatccaactt tgaacatttc agacgagttc | 2220 |
| tcctctaatg tagccaacta tcagaaggtg ggcatgcaaa agtacagcac ccttggcagt | 2280 |
| ggttcaggaa gtggatctgg cagctttgtg actgtttact ctcacctcct cctagtcgct | 2340 |
| gccggcctcg aggccccatt cctgtatcta ggaagcggct ccggtagtgg aagcggatct | 2400 |
| gtcgcagctt ttcaccaaga gtgttcccta ggcagtggga gtggttcagg cagtgggagc | 2460 |
| ggcgtgggct atcagcctta ccgggtcgtg gtcttatcct tcgagctact tggttccggc | 2520 |
| tcagggagcg gatctgggtc ttccagcgct aaaagcgctt cagtatatta ctcccaactc | 2580 |
| atgtgccagc ccatcctcct gctcggcagc ggaagcggca gtggaagcgg ttctattgtg | 2640 |
| gctgctatcg tttttatcac cctatgtgga tctggcagcg ggtctggaag tggctcctcc | 2700 |
| atagtccgct tccctaatat caccaacctc gggagcggga gcggttctgg atctggatct | 2760 |
| caggcaattg ctagcgaatt ttcatcccta ccctcttatg ccgccttcgc cacagcccag | 2820 |
| gaagcctatg agcaggcagt agctggatct ggttccggca gtgggagtgg ttcatccgtg | 2880 |
| ggccctaagc aagcctcact caacggcgtt accctcatcg gtagtggttc tggaagcggc | 2940 |
| agtgggagca tgaacgtcgc taagtatacc cagctctgcc agtatctcaa caccctaacc | 3000 |
| ctgggctccg gatctgggtc tggtagcgga tcaaaaacat atgaaagaca ttcactctcc | 3060 |
| cactttgtta atctcgacaa ccttggctcc ggtagtggct ctggctccgg ttcctttgca | 3120 |
| tatgctaacc gtaatcggtt tctatatatt atcaagctcg gatcaggcag cggcagcgga | 3180 |
| tctggaagtt ttaaggtctc tatctggaat ctcgattaca tcggatctgg cagcggaagt | 3240 |
| ggctcagggt cgaaaagcgt ctattacact tcaaacccca ccacctttca tctcgacgga | 3300 |
| gaagtgatca cgtttgacaa tctgaagaca ctcctttagg gatccagatc taacgacaaa | 3360 |
| acgacaaaac gacaaggcgc cagatctggc gtttcgtttt gtcgttttgt cgttagatct | 3420 |
| ttttccctct gccaaaaatt atggggacat catgaagccc cttgagcatc tgacttctgg | 3480 |
| ctaataaagg aaatttattt tcattgcaat agtgtgttgg aattttttgt gtctctcact | 3540 |
| cggaaggaca tatgggaggg caaatcattt aaaacatcag aatgagtatt tggtttagag | 3600 |
| tttggcaaca tatgcccatt cttccgcttc ctcgctcact gactcgctgc gctcggtcgt | 3660 |
| tcggctgcgg cgagcggtat cagctcactc aaaggcggta atacggttat ccacagaatc | 3720 |
| aggggataac gcaggaaaga acatgtgagc aaaaggccag caaaaggcca ggaaccgtaa | 3780 |
| aaaggccgcg ttgctggcgt ttttccatag getccgcccc cctgacgagc atcacaaaaa | 3840 |
| tcgacgctca agtcagaggt ggcgaaaccc gacaggacta taaagatacc aggcgtttcc | 3900 |
| ccctggaagc tccctcgtgc gctctcctgt tccgaccctg ccgcttaccg gatacctgtc | 3960 |
| cgcctttctc ccttcgggaa gcgtggcgct ttctcatagc tcacgctgta ggtatctcag | 4020 |
| ttcggtgtag gtcgttcgct ccaagctggg ctgtgtgcac gaaccccccg ttcagcccga | 4080 |
| ccgctgcgcc ttatccggta actatcgtct tgagtccaac ccggtaagac acgacttatc | 4140 |
| gccactggca gcagccactg gtaacaggat tagcagagcg aggtatgtag gcggtgctac | 4200 |
| agagttcttg aagtggtggc ctaactacgg ctacactaga agaacagtat ttggtatctg | 4260 |
| cgctctgctg aagccagtta ccttcggaaa aagagttggt agctcttgat ccggcaaaca | 4320 |
| aaccaccgct ggtagcggtg gtttttttgt ttgcaagcag cagattacgc gcagaaaaaa | 4380 |
| aggatctcaa gaagatcctt tgatcttttc tacggggtct gacgctcagt ggaacgaaaa | 4440 |
| ctcacgttaa gggattttgg tcatgagatt atcaaaaagg atcttcacct agatcctttt | 4500 |
| aaattaaaaa tgaagtttta aatcaatcta aagtatatat gagtaaactt ggtctgacag | 4560 |
| ttaccaatgc ttaatcagtg aggcacctat ctcagcgatc tgtctatttc gttcatccat | 4620 |
| agttgcctga ctcggggggg gggggcgctg aggtctgcct cgtgaagaag gtgttgctga | 4680 |
| ctcataccag gcctgaatcg ccccatcatc cagccagaaa gtgagggagc cacggttgat | 4740 |
| gagagctttg ttgtaggtgg accagttggt gattttgaac ttttgctttg ccacggaacg | 4800 |
| gtctgcgttg tcgggaagat gcgtgatctg atccttcaac tcagcaaaag ttcgatttat | 4860 |
| tcaacaaagc cgccgtcccg tcaagtcagc gtaatgctct gccagtgtta caaccaatta | 4920 |
| accaattctg attagaaaaa ctcatcgagc atcaaatgaa actgcaattt attcatatca | 4980 |
| ggattatcaa taccatattt ttgaaaaagc cgtttctgta atgaaggaga aaactcaccg | 5040 |
| aggcagttcc ataggatggc aagatcctgg tatcggtctg cgattccgac tcgtccaaca | 5100 |
| tcaatacaac ctattaattt cccctcgtca aaaataaggt tatcaagtga gaaatcacca | 5160 |
| tgagtgacga ctgaatccgg tgagaatggc aaaagcttat gcatttettt ccagacttgt | 5220 |
| tcaacaggcc agccattacg ctcgtcatca aaatcactcg catcaaccaa accgttattc | 5280 |
| attcgtgatt gcgcctgagc gagacgaaat acgcgatcgc tgttaaaagg acaattacaa | 5340 |
| acaggaatcg aatgcaaccg gcgcaggaac actgccagcg catcaacaat attttcacct | 5400 |
| gaatcaggat attcttctaa tacctggaat gotgttttcc cggggatcgc agtggtgagt | 5460 |
| aaccatgcat catcaggagt acggataaaa tgcttgatgg tcggaagagg cataaattcc | 5520 |
| gtcagccagt ttagtctgac catctcatct gtaacatcat tggcaacgct acctttgcca | 5580 |
| tgtttcagaa acaactctgg cgcatcgggc ttcccataca atcgatagat tgtcgcacct | 5640 |
| gattgcccga cattatcgcg agcccattta tacccatata aatcagcatc catgttggaa | 5700 |
| tttaatcgcg gcctcgagca agacgtttcc cgttgaatat ggctcataac accccttgta | 5760 |
| ttactgttta tgtaagcaga cagttttatt gttcatgatg atatattttt atcttgtgca | 5820 |
| atgtaacatc agagattttg agacacaacg tggctttccc ccccccccca ttattgaagc | 5880 |
| atttatcagg gttattgtct catgagcgga tacatatttg aatgtattta gaaaaataaa | 5940 |
| caaatagggg ttccgcgcac atttccccga aaagtgccac ctgacgtcta agaaaccatt | 6000 |
| attatcatga cattaaccta taaaaatagg cgtatcacga ggccctttcg tctcgcgcgt | 6060 |
| ttcggtgatg acggtgaaaa cctctgacac atgcagctcc cggagacggt cacagcttgt | 6120 |
| ctgtaagcgg atgccgggag cagacaagcc cgtcagggcg cgtcagcggg tgttggcggg | 6180 |
| tgtcggggct ggcttaacta tgcggcatca gagcagattg tactgagagt gcaccatatg | 6240 |
| cggtgtgaaa taccgcacag atgcgtaagg agaaaatacc gcatcagatt ggctat | 6296 |
| Linked hotspots (SEQ ID NO. 43): | |
| MAPRVTPLLA FSLLVLWTFP APTLGGANDA EDCCLSVTQR PIPGNIVKAF RYLLNEDGCR | 60 |
| VPAVVFTTLR GYQLCAPPDQ PWVDRIIRRL KKSSAKNKGN STRRSPVSEL KTPLGDTTHT | 120 |
| EPKSCDTPPP CPRCPGGGSS GGGSGGQPRE PQVYTLPPSR EEMTKNQVSL TCLVKGFYPS | 180 |
| DIAVEWESSG QPENNYNTTP PMLDSDGSFF LYSKLTVDKS RWQQGNIFSC SVMHEALHNR | 240 |
| FTQKSLSLSP GKGLGGLAAA TATIPIQASL PFGWLIGSGS GSGSGSYKHW PQIAQFAPSA | 300 |
| SAFFGMSRIG SGSGSGSGSF NSTYASQGLV ASIKNFKSVL YYQNNVEMGS GSGSGSGSYP | 360 |
| TLNISDEFSS NVANYQKVGM QKYSTLGSGS GSGSGSFVTV YSHLLLVAAG LEAPFLYLGS | 420 |
| GSGSGSGSVA AFHQECSLGS GSGSGSGSGV GYQPYRVVVL SFELLGSGSG SGSGSSSAKS | 480 |
| ASVYYSQLMC QPILLLGSGS GSGSGSIVAA IVFITLCGSG SGSGSGSSIV RFPNITNLGS | 540 |
| GSGSGSGSQA IASEFSSLPS YAAFATAQEA YEQAVAGSGS GSGSGSSVGP KQASLNGVTL | 600 |
| IGSGSGSGSG SMNVAKYTQL CQYLNTLTLG SGSGSGSGSK TYERHSLSHF VNLDNLGSGS | 660 |
| GSGSGSFAYA NRNRFLYIIK LGSGSGSGSG SFKVSIWNLD YIGSGSGSGS GSKSVYYTSN | 720 |
| PTTFHLDGEV ITFDNLKTLL | 740 |

### Animal Study Details

In a first immunogenicity study, C57BL/6 and BALB/c mice received immunizations with 25 μg of the test DNA plasmids on days 0, 7, 14, 21, and 28 formulated with 3% Kolliphor® polymer. Each immunization consisted of intramuscular injection of 50 μl DNA plasmid in the left and right m. tibialis anterior, respectively. Animals were sacrificed on day 56. Two groups (1-2) of mice were assigned to the following treatments:
Group 1: Plasmid P0053—pTGV4-CCL19 (Mock, i.e. DNA plasmid without the T-cell rich region), 25 μg.
Group 2: Plasmid P0057 (i.e. DNA plasmid pTGV4 with targeting unit CCL19 and the T-cell rich region RVNv0.1-S002), 25 μg.

Read-outs were body weight variation (based on 3 weekly measurements) and measurements based on spleen sampling (at day 56). Measurement performed was splenic T-cell activation (IFN-γ release ELISPOT on spleen cells restimulated with peptides derived from the selected Hotspot regions).

In a second study, K18-hACE2 C57BL/6J mice (i.e., transgenic mice carrying the human ACE-2 receptor allowing for infection by SARS-CoV-2; Bailey et al., 2020: Nature Immunology, 1327-1335, 21(11)) where immunized IM with five doses of 25 μg of the P0057 or P0053 DNA plasmids on days days 0, 7, 14, 21, and 28 formulated with 3% Kolliphor® polymer. Four mice were sacrificed at day 56 for spleen sampling for IFN-γ release ELISPOT, while 8 mice were subsequently challenged with TCID50 dose corresponding to LD50 of SARS-CoV-2 (WA1/USA 2020) at day 60. Read-outs were body weight variation (3 times/week during immunization period and daily after challenge), health scores and survival.

### Results

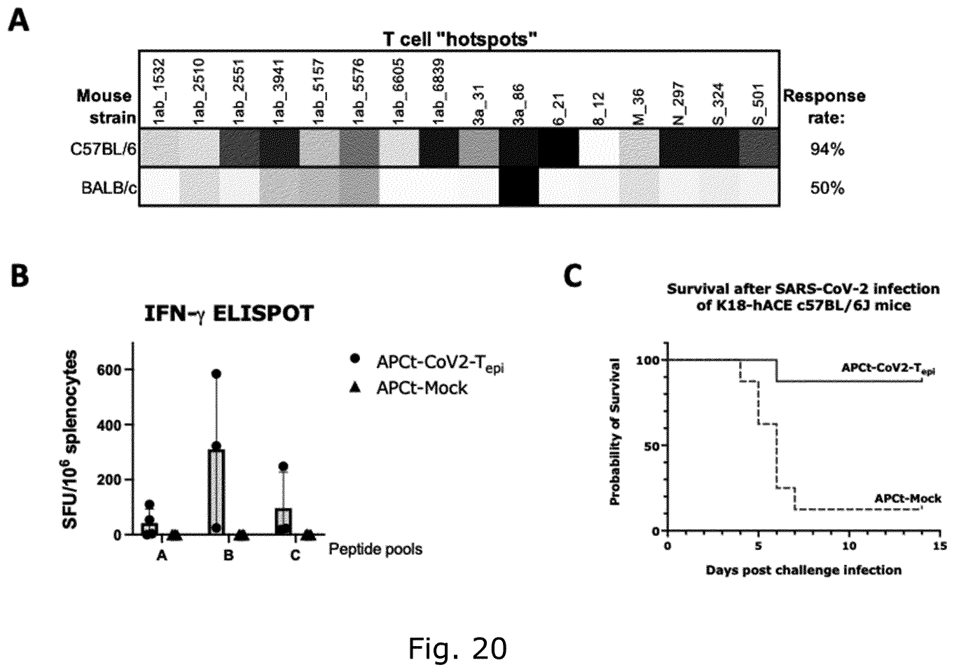

The splenocytes where restimulated with each of the hotspot T cell epitopes in a peptide format in both a pooled manner and a fully deconvoluted setup for IFN-γ ELISpot (FIGS. 20A and 20B). From the collected data it was evident that the T cell rich region generates a significant IFN-γ response when compared to stimulation with irrelevant peptides (data not shown). This is indicative of antigen specific activated CD8+/CD4+ T cells where 15 out 16 tested hotspots (response rate: 94%) had a response above baseline (a positive response was defined as the average value for irrelevant peptide stimulation+3 standard deviations) in the C57BL/6 mice to which the vaccine was designed. From FIG. 20A is can be seen that the response rate in BALB/c mice was much lower (50%) as also expected. T cell responses against the epitopes was also noted in the K18-hACE2 C57BL/6J mice (FIG. 20B), and survival data from mice immunized with P0057 (APCt-CoV2-Tepi) showed that mice were protected against lethal infections by SARS-CoV-2 (WA1/USA 2020) compared to Mock immunized mice (FIG. 20C).

### CONCLUSIONS

From the collected data it is evident that the T-cell rich designed using machine learning technology contains significant immunogenic epitopes when delivered with the APC targeting technology, and the activated T cells show capability to protect against lethal infections with SARS-CoV-2.

1. A fusion polypeptide comprising
i. a B-cell epitope-rich region, which comprises at least one fragment of at least one surface exposed protein from an intracellular pathogen, and
ii. a T-cell epitope-rich region, which comprises at least 2 densely arranged groups of T-cell inducing amino acid sequences (epitope hotspots) comprising at least one CTL inducing amino acid sequences, where the epitope hotspots are derived from at least two non-identical proteins of said intracellular pathogen,
wherein i and ii are directly fused to each other or indirectly fused to each other via linking amino acid sequences, and wherein B-cell epitopes and T-cell epitopes in said regions am derived from the intracellular pathogen.

2. The fusion polypeptide according to claim 1, which further comprises
iii. at least one amino acid sequence acting as a targeting unit for antigen presenting cells (an APC targeting unit).

3. The fusion polypeptide according to claim 2, the wherein the APC targeting unit consists of or comprises an antibody binding region with specificity for target surface molecules on antigen presenting cells, such as HLA, HLA-DP, CD14, CD40; or Toll-like receptor, such as Toll-like receptor 2.

4. The fusion polypeptide according to claim 3, wherein the APC targeting unit consists of or comprises a ligand selected from the group consisting of soluble CD40 ligand, CLEC9A peptide ligand, DEC205, FLT3L, GM-CSF, and a natural ligand, or wherein the APC targeting unit consists of or comprises a bacterial antigen.

5. The fusion polypeptide according to claim 2, which APC targeting unit targets mature dendritic cells (mDCs).

6. The fusion polypeptide according to claim 2, which APC targeting unit is selected from CCL19 and CCL21, including the human forms of CCL19 and CCL21.

7. The fusion polypeptide according to claim 2, which APC targeting unit targets the receptor CCR7.

8. (canceled)

9. (canceled)

10. (canceled)

11. (canceled)

12. (canceled)

13. (canceled)

14. (canceled)

15. (canceled)

16. (canceled)

17. (canceled)

18. The fusion polypeptide according to claim 1, which further comprises
iv. at least one amino acid sequence acting as a multimerization domain.

19. The fusion polypeptide according to claim 18, wherein the multimerization domain contributes to multimerization between copies of the fusion polypeptide through the formation of an interchain covalent bond.

20. The fusion polypeptide according to claim 19, wherein the multimerization domain is or comprises a hinge region, a dHLX protein, a hMHD2, a Collagen trimerization domain, a p53 synthetic protein, and a fibritin T4 trimerization domain, and contributes to the multimerization through the formation of an interchain covalent bond.

21. The fusion polypeptide according to claim 19, wherein the multimerization domain comprises a carboxyterminal C domain (CH3 domain), a carboxyterminal C domain of Ig (Cγ3 domain), a sequence that is substantially homologous to said C domain, and the CH3 domain of IgG3.

22. The fusion polypeptide according to claim 20, wherein a hinge region, dHLX protein, hMHD2, Collagen trimerization domain, p53 synthetic protein, fibritin T4 trimerization domain, or CH3 domains are connected by a sequence of amino acids GlyGlyGlySerSer or the amino acid sequence GlyGlyGlySerSerGlyGlyGlySerGly.

23. The fusion polypeptide according to claim 18, wherein the multimerization domain comprises a dimerization motif or any other multimerization motif, which participate in the multimerization through hydrophobic interactions.

24. The fusion polypeptide according to claim 18, wherein the multimerization domain comprises a hinge region comprising h1+h4 or h4 derived from IgG.

25. The fusion polypeptide according to claim 18 wherein the multimerization domain generally acts as a linker or as a linker between the B-cell epitope-rich region and the T-cell epitope-rich region.

26. The fusion polypeptide according to claim 1, wherein the B-cell epitope-rich region i and the T-cell epitope rich region ii, and if relevant, the APC targeting unit iii, and the multimerization domain iv are joined in any order or are joined in any order and separated by linking amino acid sequences (L).

27. The fusion polypeptide according to claim 26, wherein the fusion polypeptide has a linear structure in the N→C direction selected from:
i-L-ii,
ii-L-i,
i-L-ii-L-iii,
ii-L-i-L-iii,
i-L-iii-L-ii,
ii-L-iii-L-i,
iii-L-i-L-ii,
iii-L-ii-L-i,
i-L-ii-L-iv,
ii-L-i-L-iv,
i-L-iv-L-ii,
ii-L-iv-L-i,
iv-L-i-L-ii,
iv-L-ii-L-i,
i-L-ii-L-iii-L-iv,
i-L-ii-L-iv-L-iii,
i-L-iii-L-i-L-iv,
i-L-iii-L-iv-L-ii,
i-L-iv-L-ii-L-iii,
i-L-iv-L-iii-L-ii,
ii-L-i-L-iii-L-iv,
ii-L-i-L-iv-L-iii,
ii-L-iii-L-i-L-iv,
ii-L-iii-L-iv-L-i,
ii-L-iv-L-i-L-iii,
ii-L-iv-L-iii-L-i,
iii-L-i-L-ii-L-iv,
iii-L-i-L-iv-L-ii,
iii-L-ii-L-i-L-iv,
iii-L-ii-L-iv-L-i,
iii-L-iv-L-i-L-ii,
iii-L-iv-L-ii-L-i,
iv-L-i-L-ii-L-iii,
iv-L-i-L-ii-L-iii,
iv-L-ii-L-i-L-iii,
iv-L-ii-L-iii-L-i,
iv-L-iii-L-i-L-ii, and
iv-L-iii-L-ii-L-i,
wherein i, ii are as defined in claim 1, iii denotes at least one amino acid sequence acting as a targeting unit for antigen presenting cells (an APC targeting unit), and iv denotes at least one amino acid sequence acting as a multimerization domain, and L within each fusion polypeptide may be identical or non-identical and in each case designates a bond or a peptide linker.

28. The fusion polypeptide according to claim 1, wherein the Bcell epitope-rich region comprises non-identical fragments derived from at least two non-identical sequence variants of at least one surface exposed protein, wherein the at least 2 non-identical fragments optionally are separated with (an) amino acid linker sequence(s).

29. The fusion polypeptide according claim 1, wherein the B-cell epitope-rich region comprises
CD4⁺ epitopes (T-helper epitopes) of different variants of the surface-exposed protein found in different strains or serotypes of said pathogen, and/or CD4⁺ epitopes not found in said pathogen, or
CD4⁺ epitopes (T-helper epitopes) of different variants of the surface exposed protein found in different strains or serotypes of said pathogen, and/or CD4⁺ epitopes not found in said pathogen, and further CD8⁺ epitopes (CTL epitopes) of different variants of the surface exposed protein found in different strains or serotypes of said pathogen.

30. (canceled)

31. The fusion polypeptide according to claim 28, wherein CD4⁺ and/or CD8⁺ epitopes comprised in the B-cell epitope rich region are located to not disturb or minimally change the 3-dimensional structure of the B-cell epitopes.

32. The fusion polypeptide according to claim 1, wherein
a) the T-cell epitope-rich region comprises T-cell epitopes from other proteins of the pathogen than the surface exposed protein of i, and/or
b) the T cell epitope region comprises T-cell epitopes and/or T-cell epitope hotspots derived from at least 2 different strains or serotypes of said pathogen, and/or
c) at least some or all T-cell epitopes and/or T-cell epitope hotspots are separated by linking amino acid sequences, and/or
d) the T-cell epitope-rich region comprises amino acid sequences (pad regions), which facilitate correct antigen processing and antigen presentation of T-cell epitopes, and/or
e) the density of T-cell epitopes in the T-cell epitope hotspots is above average compared to the density of T-cell epitopes of the pathogen's protein expression products.

33. (canceled)

34. (canceled)

35. (canceled)

36. The fusion polypeptide according to claim 32, option d, wherein the number of amino acid residues in a pad region is selected from the group consisting 2, 3, 4, 5, 6, 7, 8, 9, 10, 11, 12, 13, 14, 15, 16, 17, 18, 19, 20, 21, 22, 23, 24, 25, 26, 27, 28, 29, or 30.

37. (canceled)

38. The fusion polypeptide according to claim 1, wherein the pathogen is selected from the group consisting of a virus, a protozoa, a bacterium, and a fungus.

39. (canceled)

40. The fusion polypeptide according to claim 38, wherein the virus is selected from the groups of Arenavirus, Herpesvirus, Poxvirus, Asfarviridae, Flavivirus, Alphavirus, Togavirus, Coronavirus, Hepatitis virus (A, B, C, D, or E), Orthomyxovirus, Paramyxovirus, Rhabdovirus, Bunyavirus, Filovirus, Retrovirus, Poxvirus, Adenovirus, Papillomavirus, Reovirus, Picornavirus, Calicivirus, and Astrovirus.

41. The fusion polypeptide according to claim 40, wherein the virus is SARS-Cov 1, SARS-Cov 2, MERS-COV, HCoV-229E, HCoV-NL63, HCoV-OC43, HCoV-HKU1 HIV (1 or 2), influenza virus (A, B, or C), Ebola virus, RSV, or Lassa virus

42. The fusion polypeptide according to claim 40, wherein the at least one surface exposed protein is a membrane fusion protein (MFP) or a receptor binding domain.

43. The fusion polypeptide according to claim 42, wherein the MFP is selected from a spike protein from coronavirus, a hemagglutinin, Ebola Glycoprotein (GP), Lassa Glycoprotein, HIV-1 Envelope, and an RSV Fusion glycol protein (RSV-F).

44. The fusion polypeptide according to claim 38, wherein the intracellular pathogen is a bacterium selected from
a mycobacterium species;
a Salmonella species;
a Rickettsia species;
a Chlamydia species;
Bartonella henselae;
Francisella tularensi;
Listeria monocytogenes;
a Brucella species;
a Legionella species;
a Nocardia species,
a Neisseria species;
a Yersinia species;
Shigella flexneri; and
Staphylococcus aureus,
or wherein the intracellular pathogen is a protozoa selected from
a Plasmodium species;
a Toxoplasma species;
a Cryptosporidium species;
a Leishmania species; and
Trypanosoma cruzi,
or wherein the intracellular pathogen is a fungus selected from a Pneumocystis species.

45. (canceled)

46. (canceled)

47. A nucleic acid fragment which
a) encodes the fusion polypeptide according to claim 1, or
b) encodes at least or exactly two polypeptides, of which one comprises or consists essentially of a B-cell epitope-rich region (i) as defined in claim 1, and of which one other comprises a T-cell epitope-rich region (ii) defined in claim 1.

48. The nucleic acid fragment according to claim 47, option b, wherein the B-cell epitope region (i) is fused N- or C-terminally, directly or via a linking amino acid sequence, to a multimerization domain (iv) as defined in claim 18 and/or wherein the T-cell epitope-rich region (ii) is fused N- or C-terminally, directly or via a linking amino acid sequence, to a multimerization domain (iv) as defined in claim 18.

49. The nucleic acid fragment according to claim 47, option h, wherein
A. the T-cell epitope-rich region is fused N- or C-terminally, directly or via a linking amino acid sequence, to an APC targeting unit (iii), or
B. the B-cell epitope-rich region is fused N- or C-terminally, directly or via a linking amino acid sequence, to an APC targeting unit (iii), or
C. the B-cell epitope-rich region and the T-cell epitope-rich regions are each fused N- or C-terminally, directly or via a linking amino acid sequence, to an APC targeting unit (iii),
wherein said APC targeting unit (iii) is defined in claim 2.

50. The nucleic acid fragment according to claim 47, which is tinder the control of a promoter, or wherein nucleic acid sequences encoding each polypeptide in option b is under the control of separate promoters.

51. (canceled)

52. (canceled)

53. An expression vector comprising the nucleic acid fragment according to claim 47.

54. (canceled)

55. (canceled)

56. A pharmaceutical composition comprising a fusion polypeptide according to claim 1, or comprising at least two polypeptides that can each be encoded by the nucleic acid fragment according to claim 47, option b, and further comprising a pharmaceutically acceptable carrier, vehicle, diluent, and/or excipient, and optionally an immunological adjuvant.

57. (canceled)

58. A pharmaceutical composition comprising a nucleic acid fragment according to claim 47, or an expression vector according to claim 53, or comprising
a) a first nucleic acid fragment encoding a polypeptide, which comprises or consists essentially of a B-cell epitope-rich region (i) as defined in claim 1, and a second nucleic acid fragment encoding a polypeptide which comprises or consists essentially of a T-cell epitope-rich region (ii) defined in claim 1; or
b) at least 2 expression vectors, of which one comprises the first nucleic acid fragment defined in a) and a) further comprises the second nucleic acid fragment defined in a),
and further comprising a pharmaceutically acceptable carrier, vehicle, diluent, and/or excipient, and optionally an immunological adjuvant.

59. (canceled)

60. The pharmaceutical composition according to claim 58, which further comprises an acceptable and effective amount of poloxamer 188.

61. A method of inducing or enhancing an immune response against an intracellular pathogen in an animal, including a human being, the method comprising administering to an individual in need thereof an effective and pharmaceutically acceptable amount of a fusion polypeptide according to claim 1, the nucleic acid fragment according to claim 47, an expression vector according to claim 53, or a pharmaceutical composition according to claim 56.

62. (canceled)

63. (canceled)

64. (canceled)

65. (canceled)

66. (canceled)

67. (canceled)

68. (canceled)

69. (canceled)
